SEQUENCE LISTING (10 sequences)

SEQ ID NO: 1 - protein, 9 aa, Artificial Sequence
  Synthetic peptide.
GILGFVFTL

SEQ ID NO: 2 - protein, 10 aa, Artificial Sequence
  Synthetic peptide.
ELAGIGILTV

SEQ ID NO: 3 - protein, 9 aa, Artificial Sequence
  Synthetic peptide.
TLNAWVKVV

SEQ ID NO: 4 - DNA, 36 bp, Artificial Sequence
  Synthetic oligonucleotide primer.
ggatggtgggaagatggatacagttggtgcagcatc

SEQ ID NO: 5 - DNA, 37 bp, Artificial Sequence
  Synthetic oligonucleotide primer.
gtcactggctcagggaaatagcccttgaccaggcatc

SEQ ID NO: 6 - DNA, 17 bp, Artificial Sequence
  Synthetic oligonucleotide primer.
gctagctgattaattaa

SEQ ID NO: 7 - DNA, 100 bp, Artificial Sequence
  Synthetic oligonucleotide primer.
ctagttgctggctaatggaccccaaaggctccctttcctggagaatacttctgtttctctccctggcttttgagttgtcgtacggattaattaagggccc

SEQ ID NO: 8 - protein, 61 aa, Artificial Sequence
  Synthetic peptide.
DTTEARHPHPPVTTPTTDRKGTTAEELAGIGILTVILGGKRTNNSTPTKGEFCRYPSHWRP

SEQ ID NO: 9 - protein, 10 aa, Artificial Sequence
  Synthetic peptide.
EAAGIGILTV

SEQ ID NO: 10 - protein, 9 aa, Artificial Sequence
  Synthetic peptide.
KLQCVDLHV

(19) United States
(12) Patent Application Publication
(10) Pub. No.: US 2011/0274653 A1
(43) Pub. Date: Nov. 10, 2011

(54) DENDRITIC CELL IMMUNORECEPTORS (DCIR)-MEDIATED CROSSPRIMING OF HUMAN CD8+ T CELLS

(75) Inventors: Jacques F. Banchereau, Dallas, TX (US); Eynav Klechevsky, Haifa (IL); Gerard Zurawski, Midlothian, TX (US); Sandra Zurawski, Midlothian, TX (US)

(73) Assignee: Baylor Research Institute, Dallas, TX (US)

(21) Appl. No.: 13/100,684

(22) Filed: May 4, 2011

Related U.S. Application Data
(60) Provisional application No. 61/332,465, filed on May 7, 2010.

(51) Int. Cl. A61K 38/21; A61K 39/395; A61K 38/19; A61P 35/00; A61P 31/16; A61P 31/18; A61P 35/02; A61K 35/12; A61P 37/04; A61P 31/10; A61P 31/04; A61P 33/00; A61P 33/02; A61P 37/08; A61P 31/06; A61P 3/10; A61P 29/00; A61P 19/02; A61P 25/28; A61P 17/06; A61P 31/14; A61P 31/20; A61P 31/22; A61P 27/02; A61P 25/00; A61P 43/00; A61P 27/16; A61P 7/06; A61P 1/16; A61P 11/00; C12N 5/078

(52) U.S. Cl. 424/85.5; 435/375; 424/178.1; 424/173.1; 424/85.1; 424/93.71

(57) ABSTRACT

Immunostimulatory compositions and methods comprising an ITIM motif-containing DC immunoreceptor (DCIR) to mediate potent crosspresentation are described herein. The inventors evaluated human CD8+ T cell responses generated by targeting antigens to dendritic cells (DCs) through various lectin receptors. A single exposure to a low dose of anti-DCIR-antigen conjugate initiated antigen-specific CD8+ T cell immunity by all human DC subsets including ex vivo generated DCs, skin-isolated Langerhans cells and blood mDCs and pDCs. Enhanced specific CD8+ T cell responses were observed when antigens like, FluMP, MART-1, viral (HIV gag), etc. were delivered to the DCs via DCIR, compared to those induced by a free antigen, or antigen conjugated to a control mAb or delivered via DC-SIGN, another lectin receptor. Addition of Toll-like receptor (TLR) 7/8-agonist enhanced DCIR-mediated crosspresentation as well as crosspriming. Thus, antigen targeting via the human DCIR receptor allows activation of specific CD8+ T cell immunity.

### CROSS-REFERENCE TO RELATED APPLICATIONS

This patent application is a non-provisional application of U.S. provisional patent application 61/332,465 filed on May 7, 2010 and entitled “Dendritic Cell Immunoreceptors (DCIR)-Mediated Crosspriming of Human CD8⁺ T Cells” which is hereby incorporated by reference in its entirety.

### STATEMENT OF FEDERALLY FUNDED RESEARCH

This invention was made with U.S. Government support under Contract Nos. RO-1 CA78846, RO-1 CA85540, PO-1 CA84512, and U-19 AI-57234 awarded by the NIH. The government has certain rights in this invention.

### TECHNICAL FIELD OF THE INVENTION

The present invention relates in general to the field of immunology, and more particularly, to antigen targeting via the human dendritic cell Immunoreceptors (DCIR) to mediate potent crosspresentation.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with immunostimulatory methods and compositions, including vaccines and increased effectiveness in antigen presentation.

One example of an immunostimulatory combination is taught in U.S. Pat. No. 7,387,271 issued to Noelle et al. 2008. The Noelle invention discloses an immunostimulatory composition suitable for administration to a human subject in need of immunotherapy comprising: at least one Toll-Like Receptor (TLR) agonist which is selected from the group consisting of TLR1, TLR2, TLR3, TLR4, TLR5, TLR6, TLR7, and TLR8 agonists, at least one CD40 agonist that directly binds CD40, and a pharmaceutically acceptable carrier. The TLR agonist and the CD40 agonist as described in the Noelle invention are each present in an amount such that, in combination with the other, they are effective to produce a synergistic increase in an immune response to an antigen upon administration to a human subject in need of immunotherapy.

U.S. Patent Publication No. 20080267984 (Banchereau et al. 2008) discloses compositions and methods for targeting the LOX-1 receptor on immune cells and uses for the anti-LOX-1 antibodies. The Banchereau invention includes novel compositions and methods for targeting and using anti-human LOX-1 monoclonal antibodies (mAbs) and characterized their biological functions. The anti-LOX-1 mAbs and fragments thereof are useful for the targeting, characterization, and activation of immune cells.

U.S. Patent Publication No. 20080241170 (Zurawski and Banchereau, 2008) includes compositions and methods for increasing the effectiveness of antigen presentation using a DCIR-specific antibody or fragment thereof to which an antigen is attached that forms an antibody-antigen complex, wherein the antigen is processed and presented by a dendritic cell that has been contacted with the antibody-antigen complex.

Finally, in U.S. Patent Publication No. 20080241139, filed by Delucia for an adjuvant combination comprising a microbial TLR agonist, a CD40 or 4-1BB agonist, and optionally an antigen and the use thereof for inducing a synergistic enhancement in cellular immunity. Briefly, this application is said to teach adjuvant combinations comprising at least one microbial TLR agonist such as a whole virus, bacterium or yeast or portion thereof such a membrane, spheroplast, cytoplast, or ghost, a CD40 or 4-1BB agonist and optionally an antigen wherein all 3 moieties may be separate or comprise the same recombinant microorganism or virus are disclosed. The use of these immune adjuvants for treatment of various chronic diseases such as cancers and HIV infection is also provided.

Vaccines comprising antigens attached to dendritic cells have been previously described by the present inventors. U.S. Patent Publication No. 20100135994 (Banchereau et al. 2009) discloses a HIV vaccine based on targeting maximized Gag and Nef to dendritic cells. The effectiveness of antigen presentation by an antigen presenting cell is increased by isolating and purifying a DC-specific antibody or fragment thereof to which an engineered Gag antigen is attached to form an antibody-antigen complex, wherein the Gag antigen is less susceptible to proteolytic degradation by eliminating one or more proteolytic sites; and contacting the antigen presenting cell under conditions wherein the antibody-antigen complex is processed and presented for T cell recognition. The antigen presenting cell comprises a dendritic cell and the DC-specific antibody or fragment thereof is bound to one half of a Coherin/Dockerin pair or the DC-specific antibody or fragment thereof is bound to one half of a Coherin/Dockerin pair and the engineered Gag antigen is bound to the complementary half of the Coherin/Dockerin pair to form a complex. The inventors in U.S. Patent Publication No. 20110081343 (Banchereau et al. 2009) have also described compositions and methods for targeting and delivering antigens to Langerhans cells for antigen presentation using high affinity anti-Langerin monoclonal antibodies and fusion proteins therewith.

### SUMMARY OF THE INVENTION

The present invention describes immunostimulatory compositions and methods comprising an ITIM motif-containing DC immunoreceptor (DCIR) to mediate potent crosspresentation. In a primary embodiment the present invention provides an immunostimulatory composition for generating an immune response, for a prophylaxis, a therapy or any combination thereof in a human or animal subject comprising: one or more anti-dendritic cell (DC)-specific antibodies or fragments thereof loaded or chemically coupled with one or more antigenic peptides, wherein the antigenic peptides are representative of one or more epitopes of the one or more antigens implicated or involved in a disease or a condition against which the immune response, the prophylaxis, the therapy, or any combination thereof is desired, at least one Toll-Like Receptor (TLR) agonist which is selected from the group consisting of TLR1, TLR2, TLR3, TLR4, TLR5, TLR6, TLR7, and TLR8 agonists, and a pharmaceutically acceptable carrier, wherein the conjugate and agonist are each comprised in an amount such that, in combination with the other, are effective to produce the immune response, for prophylaxis, for therapy or any combination thereof in the human or animal subject in need of immunostimulation. The composition as described herein may optionally comprise agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof.

In one aspect the anti-DC-specific antibody or fragment is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR. In another aspect the anti-DC-specific antibody is an anti-DCIR antibody selected from ATCC Accession No. PTA 10246 or PTA 10247. In another aspect the DCIR comprises an immunoreceptor tyrosine-based activation motif (ITAM).

The antigenic peptides used in the composition of the present invention comprise human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 10), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 1), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. The antigenic peptides can also comprise cancer peptides and are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. The tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67.

In yet another aspect the DC-specific antibody is humanized and the composition is administered to the human or animal subject by an oral route, a nasal route, topically or as an injection (subcutaneous, intravenous, intraperitoneal, intramuscular or intravenous)

In one embodiment the instant invention describes a vaccine comprising one or more anti-dendritic cell (DC)-specific antibodies or fragments thereof loaded or chemically coupled with one or more antigenic peptides, at least one Toll-Like Receptor (TLR) agonist which is selected from the group consisting of TLR1, TLR2, TLR3, TLR4, TLR5, TLR6, TLR7, and TLR8 agonists, and one or more optional pharmaceutically acceptable carriers and adjuvants, wherein the antibody and agonist are each comprised in an amount such that, in combination with the other, are effective to produce an immune response, for a prophylaxis, a therapy or any combination thereof in a human or an animal subject. The vaccine of the instant invention comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof.

In one aspect the anti-DC-specific antibody or fragment is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR. In another aspect the anti-DC-specific antibody is an anti-DCIR antibody selected from ATCC Accession No. PTA 10246 or PTA 10247. In another aspect the antigenic peptides comprise human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 10), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 1), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof. In yet another aspect the antigenic peptide is a cancer peptide comprising tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67. In specific aspects of the vaccine composition the DC-specific antibody is humanized and the composition is administered to the human or animal subject by an oral route, a nasal route, topically or as an injection.

In another embodiment the invention discloses a method for increasing effectiveness of antigen presentation by an antigen presenting cell comprising: (i) isolating and purifying one or more dendritic cell (DC)-specific antibody or a fragment thereof, (ii) loading or chemically coupling one or more native or engineered antigenic peptides to the DC-specific antibody to form an antibody-antigen conjugate, (iii) adding at least one Toll-Like Receptor (TLR) agonist which is selected from the group consisting of TLR1, TLR2, TLR3, TLR4, TLR5, TLR6, TLR7, and TLR8 agonists to the conjugate, and, (iv) contacting the antigen presenting cell with the conjugate and the TLR agonist wherein the antibody-antigen complex is processed and presented for T cell recognition.

The method as described above comprises the optional steps of: (i) adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the antibody-antigen conjugate and the TLR agonist prior to contacting the antigen presenting cells and (ii) measuring a level of one or more agents selected from the group consisting of IFN-γ, TNF-α, IL-12p40, IL-4, IL-5, and IL-13, wherein a change in the level of the one or more agents is indicative of the increase in the effectiveness antigen presentation by the antigen presenting cell.

In one aspect the antigen presenting cell comprises a dendritic cell (DC). In another aspect the anti-DC-specific antibody or fragment thereof is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR. In another aspect the anti-DC-specific antibody is an anti-DCIR antibody selected from ATCC Accession No. PTA 10246 or PTA 10247. In yet another aspect the antigenic peptides comprise human immunodeficiency virus (HIV) antigens and gene products selected from the group consisting of gag, pol, and env genes, the Nef protein, reverse transcriptase, string of HIV peptides (Hipo5), PSA (KLQCVDLHV)-tetramer (SEQ ID NO: 10), a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide (GILGFVFTL) tetramer (SEQ ID NO: 1), and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens or combinations and modifications thereof or cancer peptides comprising tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67. In a specific aspect the DC-specific antibody is humanized.

Yet another embodiment describes a vaccine comprising an anti-dendritic cell immunoreceptor (DCIR) monoclonal antibody conjugate, wherein the conjugate comprises the DCIR monoclonal antibody or a fragment thereof loaded or chemically coupled with one or more antigenic peptides, at least one Toll-Like Receptor (TLR) agonist which is selected from the group consisting of TLR1, TLR2, TLR3, TLR4, TLR5, TLR6, TLR7, and TLR8 agonists, and one or more optional pharmaceutically acceptable carriers and adjuvants, wherein the conjugate and agonist are each comprised in an amount such that, in combination with the other, are effective to produce an immune response, for a prophylaxis, a therapy, or any combination thereof against one or more diseases or conditions in a human or an animal subject in need thereof. The vaccine described hereinabove is adapted for use in a treatment, a prophylaxis, or a combination thereof against one or more diseases or conditions selected from influenza, HIV, cancer, and any combinations thereof in a human subject. In related aspects to the vaccine described hereinabove, the one or more antigenic peptides is a FluMP peptide (SEQ ID NO: 1), a MART-1 peptide comprising SEQ ID NO: 2, and a HIV gagp24 peptide (SEQ ID NO: 3).

In one aspect the vaccine comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof. In another aspect the vaccine further comprises an optional anti-DC-specific antibody or fragment thereof selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

The present invention further discloses a method for a treatment, a prophylaxis, or a combination thereof against one or more diseases or conditions in a human subject comprising the steps of: identifying the human subject in need of the treatment, the prophylaxis or a combination thereof against the one or more diseases or conditions and administering a vaccine composition comprising: (i) an anti-dendritic cell immunoreceptor (DCIR) monoclonal antibody conjugate, wherein the conjugate comprises the DCIR monoclonal antibody or a fragment thereof loaded or chemically coupled with one or more antigenic peptides, wherein the antigenic peptides are representative of one or more epitopes of the one or more antigens implicated or involved in the one or more diseases or conditions against which the prophylaxis, the therapy, or both is desired, (ii) at least one Toll-Like Receptor (TLR) agonist selected from the group consisting of TLR1, TLR2, TLR3, TLR4, TLR5, TLR6, TLR7, and TLR8 agonists, and (iii) one or more optional pharmaceutically acceptable carriers and adjuvants, wherein the conjugate and agonist are each comprised in an amount such that, in combination with the other, are effective to produce an immune response, for the prophylaxis, the therapy or any combination thereof against the one or more diseases or conditions in the human subject.

The one or more diseases or conditions treated by the method disclosed hereinabove comprises influenza, cancer, HIV, or any combinations thereof, wherein the cancers are selected from the group consisting of leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

In one aspect the vaccine comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof. In another aspect the vaccine is administered to the human subject by an oral route, a nasal route, topically or as an injection. In yet another aspect the vaccine further comprises an optional anti-DC-specific antibody or a fragment thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor, and IL-2 receptor, ICAM-1, Fcγ receptor, LOX-1, and ASPGR. In related aspects to the vaccine described in the method hereinabove, the one or more antigenic peptides is a FluMP peptide (SEQ ID NO: 1), a MART-1 peptide comprising SEQ ID NO: 2, and a HIV gagp24 peptide (SEQ ID NO: 3).

The present invention also provides a method for increasing effectiveness of antigen presentation by one or more dendritic cells (DCs) in a human subject comprising the steps of: isolating one or more DCs from the human, exposing the isolated DCs to activating amounts of a composition or a vaccine comprising an anti-dendritic cell immunoreceptor (DCIR) monoclonal antibody conjugate, wherein the conjugate comprises the DCIR monoclonal antibody or fragments thereof loaded or chemically coupled with one or more antigenic peptides, at least one Toll-Like Receptor (TLR) agonist which is selected from the group consisting of TLR1, TLR2, TLR3, TLR4, TLR5, TLR6, TLR7, and TLR8 agonists and a pharmaceutically acceptable carrier to form an activated DC complex, and reintroducing the activated DC complex into the human subject. The method further comprises the optional steps of measuring a level of one or more agents selected from the group consisting of IFN-γ, TNF-α, IL-12p40, IL-4, IL-5, and IL-13, wherein a change in the level of the one or more agents is indicative of the increase in the effectiveness of the one or more DCs and the step of adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the conjugate and the TLR agonist prior to exposing the DCs.

In one aspect the method further comprises the step of adding one or more anti-DC-specific antibody or fragment thereof selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR. In another aspect of the method the antigenic peptides comprise human immunodeficiency virus (HIV) antigens and gene products, one or more cancer peptides and tumor associated antigens, or both.

The present invention further provides a method of providing immunostimulation by activation of one or more dendritic cells (DCs) to a human subject for a prophylaxis, a therapy or a combination thereof against one or more viral, bacterial, fungal, parasitic, protozoal, parasitic diseases, and allergic disorders comprising the steps of: (i) identifying the human subject in need of immunostimulation for the prophylaxis, the therapy or a combination thereof against the viral, bacterial, fungal, parasitic, protozoal, parasitic diseases, and allergic disorders, (ii) isolating one or more DCs from the human subject, (iii) exposing the isolated DCs to activating amounts of a composition or a vaccine comprising an anti-dendritic cell immunoreceptor (DCIR) monoclonal antibody conjugate, wherein the conjugate comprises the DCIR monoclonal antibody or fragments thereof loaded or chemically coupled with one or more antigenic peptides, at least one Toll-Like Receptor (TLR) agonist which is selected from the group consisting of TLR1, TLR2, TLR3, TLR4, TLR5, TLR6, TLR7, and TLR8 agonists and a pharmaceutically acceptable carrier to form an activated DC complex, and (iv) reintroducing the activated DC complex into the human subject. The immunostimulation method further comprising the optional step of measuring a level of one or more agents selected from the group consisting of IFN-γ, TNF-α, IL-12p40, IL-4, IL-5, and IL-13, wherein a change in the level of the one or more agents is indicative of the immunostimulation.

In one aspect the method further comprises the step of adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the conjugate and the TLR agonist prior to exposing the DCs.

In another aspect the method further comprises the step of adding one or more optional anti-DC-specific antibody or fragment thereof selected from an antibody that specifically binds to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

The antigenic peptides may comprise bacterial antigens selected from pertussis toxin, filamentous hemagglutinin, pertactin, FIM2, FIM3, adenylate cyclase and other pertussis bacterial antigen components, diptheria bacterial antigens, diptheria toxin or toxoid, other diptheria bacterial antigen components, tetanus bacterial antigens, tetanus toxin or toxoid, other tetanus bacterial antigen components, streptococcal bacterial antigens, gram-negative bacilli bacterial antigens, Mycobacterium tuberculosis bacterial antigens, mycolic acid, heat shock protein 65 (HSP65), Helicobacter pylori bacterial antigen components; pneumococcal bacterial antigens, haemophilus influenza bacterial antigens, anthrax bacterial antigens, and rickettsiae bacterial antigens, fungal antigens selected from candida fungal antigen components, histoplasma fungal antigens, cryptococcal fungal antigens, coccidiodes fungal antigens and tinea fungal antigens, protozoal and parasitic antigens selected from plasmodium falciparum antigens, sporozoite surface antigens, circumsporozoite antigens, gametocyte/gamete surface antigens, blood-stage antigen pf 155/RESA, toxoplasma, schistosomae antigens, leishmania major and other leishmaniae antigens and trypanosoma cruzi antigens, antigens involved in autoimmune diseases, allergy, and graft rejection selected from diabetes, diabetes mellitus, arthritis, multiple sclerosis, myasthenia gravis, systemic lupus erythematosis, autoimmune thyroiditis, dermatitis, psoriasis, Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis, and antigens involved in allergic disorders selected from Japanese cedar pollen antigens, ragweed pollen antigens, rye grass pollen antigens, animal derived antigens, dust mite antigens, feline antigens, histocompatiblity antigens, and penicillin and other therapeutic drugs. In yet another aspect the DC-specific antibody is humanized.

### BRIEF DESCRIPTION OF THE DRAWINGS

For a more complete understanding of the features and advantages of the present invention, reference is now made to the detailed description of the invention along with the accompanying figures and in which:

FIGS. 1A-1D show cellular distribution of DCIR: (FIG. 1A) Flow cytometry analysis of DCIR expression on peripheral blood mononuclear cells. Circulating mononuclear cells were stained with 10 μg/ml anti-DCIR mAb followed by PE-conjugated goat anti-mouse IgG. Cells were incubated with FITC-conjugated anti-CD19, anti-CD4, anti-CD8 (for lymphocytes), anti-CD16, anti-CD56 (for NK cells), anti-CD14 mAb (for monocytes) or with anti-CD11c, anti-HLA-DR and anti-CD123 mAb (for pDCs or mDCs) and analyzed by flow cytometry. Data presented are representative of three independent runs performed on three different donors, (FIG. 1B) Expression analysis of DCIR by flow cytometry on skin-derived DC subsets: epidermal LCs, dermal CD1a⁺ DCs and dermal CD14⁺ DCs, (FIG. 1C) Human epidermal sheets were stained with anti-DCIR and analyzed by fluorescence microscopy, revealed the expression of DCIR on HLA-DR⁺ LCs, (FIG. 1D) Expression analysis of DCIR by flow cytometry on CD34⁺-derived DC subsets CD1a⁺ LCs and CD14⁺ DCs;

FIG. 2A shows I: diagram of mouse IgG1 crosslinked to the target antigen FluMP, II-III: diagram of chimeric mAbs (IgG4).doc conjugated to coh.antigen (FluMP II or MART-1 III), IV-V: diagram of chimeric fusion mAb IgG4-antigen (HIV gag MART-1 IV or p24 V);

FIGS. 2B and 2C show the crosspresentation of FluMP protein by anti-DCIR conjugate mAb: (FIG. 2B) Enhanced crosspresentation of FluMP to CD8⁺ T cells by CD1a⁺ LCs cultured with chemically cross-linked anti-DCIR-FluMP, crosslinked control IgG-FluMP proteins, or free FluMP. Dot plots show the proportions of HLA-A201-FluMP (58-66) peptide tetramer-positive CD8⁺ T cells. Data are representative of three independent studies, (FIG. 2C) Shows the percentage of FluMP-specific CD8⁺ T cells in response to targeting with decreasing concentrations of crosslinked mAb-FluMP constructs or free FluMP. Graph shows mean of duplicate;

FIGS. 2D and 2E show the engineering and characterization of targeted proteins into DCIR mAb: (FIG. 2D) SDS-PAGE-reducing gel of mouse anti-DCIR mAbs (clone 9E8 and 24A5), chimeric mouse/human anti-DCIR (IgG4) and control IgG4 fused to a Dockerin domain (mAb.Doc). FluMP and MART-1 fused to a cohesin domain (coh.FluMP and coh.MART-1), and the fusion proteins anti-DCIR-p24 and control IgG4-p24. The gel was stained with comassee blue. The molecular weights of the proteins are indicated on the left of the figure, (FIG. 2E) Binding analysis of anti-DCIR.doc-coh.FluMP complex mAb to monocyte-derived DCs. Day 6 immature GM-IL4 DCs were treated with 50 nM of biotinylated anti-DCIR-FluMP, and control IgG4-FluMP conjugate mAbs. The complexs were detected with a phycoerythrin-conjugated streptavidin. The anti-DCIR.doc-coh.FluMP complex mAb bound the DCs (black histogram), while the respective control conjugate mAb did not bind to DCs (gray histogram);

FIG. 2F represents staining of HLA-A201-FluMP complexes on CD34⁺-derived DCs unpulsed (control DCs, gray histogram), or pulsed with 50 nM DCIR-targeted FluMP. Cells were activated with 5 μg/ml anti-CD40 mAb (12E12, Baylor Research Institute; BIIR) and stained after 24 h with PE-labeled tetramerized anti-HLA-A201-FluMP Fab (M1D12)⁵⁰;

FIG. 2G shows the crosspresentation of FluMP to CD8⁺ T cells by autologous HLA-A201⁺ CD34⁺-derived LCs that were cultured with 8 nM (upper panel) or 0.8 nM (lower panel) of anti-DCIR.doc-coh.FluMP or IgG4.doc-coh.FluMP conjugate mAbs. Dot plots show the proportions of HLA-A201-FluMP (58-66) peptide tetramer-positive CD8⁺ T cells after 10 days;

FIG. 2H is a graphical representation of the proportions of HLA-A201-FluMP (58-66) tetramer-positive-CD8⁺ T cells induced by DCs that were pulsed for 18 h with 8 nM anti-DCIR.doc-coh.FluMP or control IgG2a.doc-coh.FluMP conjugate mAbs washed and cultured with autologous CD8⁺ T cells for 10 days. Graphs show the proportions of HLA-A201-FluMP (58-66) tetramer-positive CD8⁺ T cells, mean±sd, N=3;

FIGS. 3A and 3B show that DCIR allows crosspresentation of proteins by LCs: (FIG. 3A) Skin-derived LCs from an HLA-A201⁺ donor were targeted with 8 nM each of anti-DCIR.doc-coh.FluMP or IgG4.doc-coh.FluMP conjugate mAbs, matured with CD40L and co-cultured with autologous CD8⁺ T cells. 10 days later, CD8⁺ T cell expansion was evaluated by specific HLA-A201-FluMP (58-66) tetramer staining Data are representative of two independent experiments performed with cells from two different donors, (FIG. 3B) IFN-γ levels as measured by Luminex in the culture supernatant of CD8⁺ T cells expanded for 10 days by autologous skin LCs targeted with anti-DCIR.doc-coh.FluMP or IgG4.doc-coh.FluMP conjugate mAbs. Graph shows mean±sd, N=3;

FIGS. 4A to 4C show that DCIR is a global target for all blood DC subsets: (FIG. 4A) Blood-derived mDCs from an HLA-A201 donor are targeted with 8 nM, 0.8 nM or 80 pM each of anti-DCIR.doc-coh.FluMP (clone 24A5), IgG4.doc-coh.FluMP conjugate mAbs, or free coh.FluMP, matured with CD40L and co-cultured with autologous CD8⁺ T cells. 10 days later, CD8⁺ T cell expansion was evaluated by specific HLA-A201-FluMP (58-66) tetramer staining Data are representative of three independent studies, (FIG. 4B) Blood-derived pDCs from an HLA-A201 donor were targeted with 8 nM, 0.8 nM or 80 pM each of anti-DCIR.doc-coh.FluMP (clone 24A5), IgG4.doc-coh.FluMP, or free coh.FluMP, matured with CD40L and co-cultured with autologous CD8⁺ T cells. 10 days later, T cell expansion was evaluated by specific HLA-A201-FluMP (58-66) tetramer staining Data are representative of three independent studies, (FIG. 4C) Percentage of FluMP-specific CD8⁺ T cells induced by 8 nM DCIR.doc-coh.FluMP complex mAb-targeted mDCs or pDCs. Graph shows results of 3 independent studies using 2 different clones of DCIR mAb p=0.02;

FIGS. 5A to 5D show the crosspriming of Mart-1 and HIV gag p24 protein by anti-DCIR fusion mAb: (FIG. 5A) Skin-derived LCs from an HLA-A201⁺ donor were purified and cultured for 10 days with autologous purified T cells in the presence of 30 nM anti-DCIR.doc-coh.MART-1 or IgG4.doc-coh.MART-1 conjugate mAbs. DCs were activated with CD40L. MART-1-specific CD8⁺ T cells expansion was measured with a specific HLA-A201-MART-1 (26-35) tetramer; (FIG. 5B) Anti-DCIR-MART-1 or IgG4-MART-1 (25 nM) fusion proteins were used to target monocyte-derived IFN-α DCs. DCs were activated with CD40L and cultured with naïve autologous CD8⁺ T cells. After 10 days, cells were restimulated for 24 h with fresh DCs loaded with peptides derived from MART-1 protein or with unloaded DCs as a control. Plot shows the percentage of primed CD8⁺ T cells coexpressing IFN-γ and CD107a in response to a specific MART-1 peptide cluster, (FIG. 5C) CD34⁺-derived LCs were targeted with DCIR-MART-1 or control IgG4-MART-1 fusion proteins and cultured with naïve CD8⁺ T cells for 9 days. Graph shows the percentage of cells coexpressing Granzyme B and perforin as analyzed at the end of the culture by flow cytometry, (FIG. 5D) Anti-DCIR-p24 or control IgG4-p24 (25 nM) fusion proteins were used to target CD34⁺-derived LCs. DCs were activated with CD40L and cultured with naïve autologous CD8⁺ T cells. After 2 consecutive stimulations, the proliferated cells were sorted and restimulated for 24 h with fresh LCs and HIV gag p24 protein to evaluate IFN-γ secretion by Luminex. Cells with no protein served as a control. Values are average of duplicates. Data are representative of two independent studies;

FIGS. 6A to 6C show TLR7/8-signaling enhances DCIR-mediated secondary CD8⁺ T cell response by mDCs: (FIG. 6A) Blood-derived mDCs from an HLA-A201⁺ donor were targeted with 12 nM, 2 nM or 200 pM of anti-DCIR.doc-coh.FluMP complex mAb, activated with either TLR3 TLR4 or TLR7/8-agonists (Poly I:C, LPS or CL075) and co-cultured with autologous CD8⁺ T cells for 10 days. Graph shows the percentage of FluMP-specific CD8⁺ T cells measured with a specific HLA-A201-FluMP (58-66) tetramer for each amount of anti-DCIR.doc-coh.FluMP complex mAb and with each DC-activator tested. DCs with no activation were used as a control (No activation-(--), TLR7/8-(♦) TLR3-(*), TLR4-(∘), agonists; CL075, Poly I:C and LPS, respectively). Data are representative of four independent experiments with four different donors. The graph shows mean±s.d, N=3; (FIG. 6B) Shows blood-derived mDCs from an HLA-A201⁺ donor were targeted with 8 nM of anti-DCIR.doc-coh.FluMP or IgG4.doc-coh.FluMP complex mAb, activated with either TLR7/8-, TLR3-, TLR4-agonists (CL075, Poly I:C and LPS, respectively) and co-cultured with autologous CD8⁺ T cells for 10 days. Graph shows the percentage of FluMP-specific CD8⁺ T cells as measured with a specific HLA-A201-FluMP (58-66) tetramer. Conditions indicated in the graph are: No activation; CL075 1 μg/ml; Poly I:C 10 μg/ml; LPS 50 ng/ml. The graph shows mean±s.d, N=3, (FIG. 6C) Same study as in 6B. Graph shows the mean percentage of FluMP-specific CD8⁺ T cells as measured with a specific HLA-A201-FluMP (58-66) tetramer. Conditions indicated in the graph are: No activation; CL075—0.2 μg/ml and 2 μg/ml; Poly I:C—5 μg/ml and 25 μg/ml; LPS—10 ng/ml and 100 ng/ml;

FIGS. 7A to 7G show that TLR7/8-signaling enhances DCIR-mediated primary CD8⁺ T cell response by mDCs: (FIG. 7A) IFNα-DCs from an HLA-A201⁺ donor were targeted with 17 nM of anti-DCIR-MART-1 or a control IgG4-MART-1 fusion proteins, activated with either CD40L (100 ng/ml), CL075 (1 μg/ml), Poly I:C (5 μg/ml) or LPS (50 ng/ml) and co-cultured with autologous naïve CD8⁺ T cells for 10 days. The expansion of MART-1-specific CD8⁺ T cells was measured with a specific HLA-A201-MART-1 (26-35) tetramer. Data are of two independent experiments with two different donors, (FIG. 7B) Blood-derived mDCs from an HLA-A201⁺ donor were targeted with 30 nM of anti-DCIR-MART-1 fusion protein or anti-DCIR-p24, activated with either CD40L or TLR7/8-agonists and co-cultured with autologous naïve CD8⁺ T cells for 10 days. Upper panel shows the proportions of HLA-A201-MART-1 (26-35) peptide tetramer-positive CD8⁺ T cells expanded by purified blood mDCs cultured with anti-DCIR-MART-1 fusion protein and activated with either CD40L or TLR7/8-agonist. Lower panel shows the proportions of HLA-A201-HIV gag p24 (151-159) peptide tetramer-positive CD8⁺ T cells expanded by purified blood mDCs targeted with anti-DCIR-p24 fusion protein and activated with either CD40L or TLR7/8-agonist. Data are of two independent studies with two different donors, (FIG. 7C) Shows the expression of intracellular effector molecules Granzyme B and perforin was assessed by flow cytometry on CD8⁺ T cells primed by IFNα-DCs-targeted with 10 nM of anti-DCIR-MART-1 or IgG4-MART-1 fusion proteins and activated with CD40L, CL075 or a combination of CD40L and CL075. The expression on the antigen specific MART-1 (26-35)-positive cells was analyzed by co staining with the corresponding HLA-A201-tetramer. Data are representative of two independent studies, (FIG. 7D) Shows the frequency of MART-1-specific CD8⁺ T cells, as measured with a specific HLA-A201-MART-1 (26-35) tetramer, after expansion with anti-DCIR-MART-1-targeted DCs, control IgG4-MART-1 or no antigen that were activated with CD40L, TLR7/8-ligand or a combination of CD40L and TLR7/8-ligand. Each dot represent a single study, (FIG. 7E) Upper panel: IFNα-DCs were targeted with 17 nM of anti-DCIR-MART-1 or a control IgG4-MART-1 fusion proteins, activated with either CD40L (100 ng/ml), CL075 (1 μg/ml), Poly I:C (10 μg/ml) or LPS (50 ng/ml) and co-cultured with autologous naïve CD8⁺ T cells. 10 days later, cells were restimulated with fresh DCs that were loaded with 15mer overlapping peptides-derived from the MART-1 protein. Plots show the level of intracytoplasmic IFN-γ by CD8⁺ T cells after 5 h stimulation in the presence of monensin. Lower panel: anti-DCIR-p24 or a control IgG4-p24 fusion proteins were used as a model antigen. (FIG. 7F) IFNα-DCs were targeted with 113 nM of anti-DCIR-MART-1 fusion protein activated with either CD40L (100 ng/ml) or CL075 (1 μg/ml) and co-cultured with autologous naïve CD8⁺ T cells. 10 days later, cells were restimulated with fresh DCs that were loaded with 15mer overlapping peptides-derived from the MART-1 protein. The levels of IL-4, IL-5, IL-13, IFN-γ, TNF-α and IL-12p40 were measured by Luminex in the culture supernatant after 24 h. The graph shows mean±s.d, N=3, (FIG. 7G) IFNα-DCs were targeted with 10 nM of anti-DCIR-MART-1 fusion protein, activated with either CD40L (100 ng/ml) or CL075 (1 μg/ml), or a combination of CD40L and CL075 and co-cultured with autologous naïve CD8⁺ T cells. 10 days later, cells were restimulated with fresh DCs that were loaded with 15mer overlapping peptides-derived from the MART-1 protein or with unloaded DCs. Plots show the level of intracytoplasmic IFN-γ and TNF-α by CD8⁺ T cells after 5 h stimulation in the presence of monensin;

FIGS. 8A to 8C shows that the anti-DCIR antibody fails to deliver inhibitory signals to human DCs: (FIGS. 8A and 8B) Illustrative flow cytometry data showing the expression of CD86 on the surface of DCIR-ligated- or control-CD1a⁺ LCs in the presence or absence of CD40L, (FIG. 8C) Luminex assay for IL-6 was performed on supernatants from DCIR or control ligated-skin DC subsets activated for 24 h with CD40L or TLR7/8-agonist. One of two independent studies is shown; and

FIGS. 9A to 9D show that DCIR ligation does not inhibit CD8⁺ T cell priming: (FIG. 9A) DCIR-ligated DCs induce a similar level of allogeneic CD8⁺ T cell proliferation compared to control DCs, as determined by [³H]-thymidine incorporation in the presence or absence of CD40 activation. The graph shows mean±s.d, N=3, (FIG. 9B) Flow cytometry analysis of the expression of PD-1, CTLA-4 or CD28 on allogeneic CD8⁺ T cells primed by DCIR-ligated DCs (blue line) or control DCs (red line), (FIG. 9C) Graphs show the level of cytokine secretion IFN-γ, IL-2, TNF-α and IL-10 by activated CD8⁺ T cells that were primed by allogeneic DCIR-ligated DCs or control DCs. Cytokines were measured in response to anti-CD3/CD28 microbeads stimulation and analysed after 24 h by Luminex, (FIG. 9D) Expression of effector molecules: Granzyme A, Granzyme B and perforin, as evaluated by flow cytometry (right panel) on MART-1-specific CD8⁺ T cells that were primed by DCIR-ligated- or control-MART-1 peptide-loaded LCs. Data are representative of three independent studies.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The invention includes also variants and other modification of an antibody (or “Ab”) of fragments thereof, e.g., anti-CD40 fusion protein (antibody is used interchangeably with the term “immunoglobulin”). As used herein, the term “antibodies or fragments thereof,” includes whole antibodies or fragments of an antibody, e.g., Fv, Fab, Fab′, F(ab′)₂, Fc, and single chain Fv fragments (ScFv) or any biologically effective fragments of an immunoglobulins that binds specifically to, e.g., CD40. Antibodies from human origin or humanized antibodies have lowered or no immunogenicity in humans and have a lower number or no immunogenic epitopes compared to non-human antibodies. Antibodies and their fragments will generally be selected to have a reduced level or no antigenicity in humans.

As used herein, the terms “Ag” or “antigen” refer to a substance capable of either binding to an antigen binding region of an immunoglobulin molecule or of eliciting an immune response, e.g., a T cell-mediated immune response by the presentation of the antigen on Major Histocompatibility Antigen (MHC) cellular proteins. As used herein, “antigen” includes, but is not limited to, antigenic determinants, haptens, and immunogens which may be peptides, small molecules, carbohydrates, lipids, nucleic acids or combinations thereof. The skilled immunologist will recognize that when discussing antigens that are processed for presentation to T cells, the term “antigen” refers to those portions of the antigen (e.g., a peptide fragment) that is a T cell epitope presented by MHC to the T cell receptor. When used in the context of a B cell mediated immune response in the form of an antibody that is specific for an “antigen”, the portion of the antigen that binds to the complementarity determining regions of the variable domains of the antibody (light and heavy) the bound portion may be a linear or three-dimensional epitope. In the context of the present invention, the term antigen is used on both contexts, that is, the antibody is specific for a protein antigen (CD40), but also carries one or more peptide epitopes for presentation by MHC to T cells. In certain cases, the antigens delivered by the vaccine or fusion protein of the present invention are internalized and processed by antigen presenting cells prior to presentation, e.g., by cleavage of one or more portions of the antibody or fusion protein.

As used herein, the term “conjugate” refers to a protein having one or more targeting domains, e.g., an antibody, and at least one antigen, e.g., a small peptide or a protein. These conjugates include those produced by recombinant methods such as fusion proteins, those produced by chemical methods, such as by chemical coupling, for example, coupling to sulfhydryl groups, and those produced by any other method whereby one or more antibody targeting domains and at least one antigen, are linked, directly or indirectly via linker(s) to a targeting agent. An example of a linker is a cohesin-dockerin (coh-doc) pair, a biotin-avidin pair, histidine tags bound by Zn, and the like.

Examples of viral antigens for use with the present invention include, but are not limited to, e.g., HIV, HCV, CMV, adenoviruses, retroviruses, picornaviruses, etc. Non-limiting example of retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens. The at least one viral antigen may be peptides from an adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus. In certain specific, non-limiting examples, the at least one viral antigen are peptides obtained from at least one of HIV, CMV, hepatitis A, B, and C, influenza, measles, polio, smallpox, rubella; respiratory syncytial, herpes simplex, varicella zoster, Epstein-Barr, Japanese encephalitis, rabies, flu, and/or cold viruses.

Bacterial antigens for use with the DCIR disclosed herein include, but are not limited to, e.g., bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; small pox; allergies and cancers.

Fungal antigens for use with compositions and methods of the invention include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Examples of protozoal and other parasitic antigens include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

Target antigens on cell surfaces for delivery include those characteristic of tumor antigens typically will be derived from the cell surface, cytoplasm, nucleus, organelles and the like of cells of tumor tissue. Examples of tumor targets for the antibody portion of the present invention include, without limitation, hematological cancers such as leukemias and lymphomas, neurological tumors such as astrocytomas or glioblastomas, melanoma, breast cancer, lung cancer, head and neck cancer, gastrointestinal tumors such as gastric or colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, and leukemia.

Examples of antigens that may be delivered alone or in combination to immune cells for antigen presentation using the present invention includes tumor proteins, e.g., mutated oncogenes; viral proteins associated with tumors; and tumor mucins and glycolipids. The antigens may be viral proteins associated with tumors would be those from the classes of viruses noted above. Certain antigens may be characteristic of tumors (one subset being proteins not usually expressed by a tumor precursor cell), or may be a protein that is normally expressed in a tumor precursor cell, but having a mutation characteristic of a tumor. Other antigens include mutant variant(s) of the normal protein having an altered activity or subcellular distribution, e.g., mutations of genes giving rise to tumor antigens.

Antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor.

Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

As used herein, the term “antigenic peptide” refers to that portion of a polypeptide antigen that is specifically recognized by either B-cells or T-cells. B-cells respond to foreign antigenic determinants via antibody production, whereas T-lymphocytes are the mediate cellular immunity. Thus, antigenic peptides are those parts of an antigen that are recognized by antibodies, or in the context of an MHC, by T-cell receptors.

As used herein, the term “epitope” refers to any protein determinant capable of specific binding to an immunoglobulin or of being presented by a Major Histocompatibility Complex (MHC) protein (e.g., Class I or Class II) to a T-cell receptor. Epitopic determinants are generally short peptides 5-30 amino acids long that fit within the groove of the MHC molecule that presents certain amino acid side groups toward the T cell receptor and has certain other residues in the groove, e.g., due to specific charge characteristics of the groove, the peptide side groups and the T cell receptor. Generally, an antibody specifically binds to an antigen when the dissociation constant is 1 mM, 100 nM, or even 10 nM.

As used herein, the term “vector” is used in two different contexts. When using the term “vector” with reference to a vaccine, a vector is used to describe a non-antigenic portion that is used to direct or deliver the antigenic portion of the vaccine. For example, an antibody or fragments thereof may be bound to or form a fusion protein with the antigen that elicits the immune response. For cellular vaccines, the vector for delivery and/or presentation of the antigen is the antigen presenting cell, which is delivered by the cell that is loaded with antigen. In certain cases, the cellular vector itself may also process and present the antigen(s) to T cells and activate an antigen-specific immune response. When used in the context of nucleic acids, a “vector” refers a construct, which is capable of delivering, and preferably expressing, one or more genes or polynucleotide sequences of interest in a host cell. Examples of vectors include, but are not limited to, viral vectors, naked DNA or RNA expression vectors, DNA or RNA expression vectors associated with cationic condensing agents, DNA or RNA expression vectors encapsulated in liposomes, and certain eukaryotic cells, such as producer cells.

As used herein, the terms “stable” and “unstable” when referring to proteins is used to describe a peptide or protein that maintains its three-dimensional structure and/or activity (stable) or that loses immediately or over time its three-dimensional structure and/or activity (unstable). As used herein, the term “insoluble” refers to those proteins that when produced in a cell (e.g., a recombinant protein expressed in a eukaryotic or prokaryotic cell or in vitro) are not soluble in solution absent the use of denaturing conditions or agents (e.g., heat or chemical denaturants, respectively). The antibody or fragment thereof and the linkers taught herein have been found to convert antibody fusion proteins with the peptides from insoluble and/or unstable into proteins that are stable and/or soluble. Another example of stability versus instability is when the domain of the protein with a stable conformation has a higher melting temperature (Tₘ) than the unstable domain of the protein when measured in the same solution. A domain is stable compared to another domain when the difference in the Tₘ is at least about 2° C., more preferably about 4° C., still more preferably about 7° C., yet more preferably about 10° C., even more preferably about 15° C., still more preferably about 20° C., even still more preferably about 25° C., and most preferably about 30° C., when measured in the same solution.

As used herein, “polynucleotide” or “nucleic acid” refers to a strand of deoxyribonucleotides or ribonucleotides in either a single- or a double-stranded form (including known analogs of natural nucleotides). A double-stranded nucleic acid sequence will include the complementary sequence. The polynucleotide sequence may encode variable and/or constant region domains of immunoglobulin that are formed into a fusion protein with one or more linkers. For use with the present invention, multiple cloning sites (MCS) may be engineered into the locations at the carboxy-terminal end of the heavy and/or light chains of the antibodies to allow for in-frame insertion of peptide for expression between the linkers. As used herein, the term “isolated polynucleotide” refers to a polynucleotide of genomic, cDNA, or synthetic origin or some combination thereof. By virtue of its origin the “isolated polynucleotide” (1) is not associated with all or a portion of a polynucleotide in which the “isolated polynucleotides” are found in nature, (2) is operably linked to a polynucleotide which it is not linked to in nature, or (3) does not occur in nature as part of a larger sequence. The skilled artisan will recognize that to design and implement a vector can be manipulated at the nucleic acid level by using techniques known in the art, such as those taught in Current Protocols in Molecular Biology, 2007 by John Wiley and Sons, relevant portions incorporated herein by reference. Briefly, the encoding nucleic acid sequences can be inserted using polymerase chain reaction, enzymatic insertion of oligonucleotides or polymerase chain reaction fragments in a vector, which may be an expression vector. To facilitate the insertion of inserts at the carboxy terminus of the antibody light chain, the heavy chain, or both, a multiple cloning site (MCS) may be engineered in sequence with the antibody sequences.

As used herein, the term “polypeptide” refers to a polymer of amino acids and does not refer to a specific length of the product; thus, peptides, oligopeptides, and proteins are included within the definition of polypeptide. This term also does not refer to or exclude post expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. The term “domain,” or “polypeptide domain” refers to that sequence of a polypeptide that folds into a single globular region in its native conformation, and that may exhibit discrete binding or functional properties.

A polypeptide or amino acid sequence “derived from” a designated nucleic acid sequence refers to a polypeptide having an amino acid sequence identical to that of a polypeptide encoded in the sequence, or a portion thereof wherein the portion consists of at least 3-5 amino acids, preferably at least 4-7 amino acids, more preferably at least 8-10 amino acids, and even more preferably at least 11-15 amino acids, or which is immunologically identifiable with a polypeptide encoded in the sequence. This terminology also includes a polypeptide expressed from a designated nucleic acid sequence.

As used herein, “pharmaceutically acceptable carrier” refers to any material that when combined with an immunoglobulin (Ig) fusion protein of the present invention allows the Ig to retain biological activity and is generally non-reactive with the subject's immune system. Examples include, but are not limited to, standard pharmaceutical carriers such as a phosphate buffered saline solution, water, emulsions such as an oil/water emulsion, and various types of wetting agents. Certain diluents may be used with the present invention, e.g., for aerosol or parenteral administration, that may be phosphate buffered saline or normal (0.85%) saline.

Dendritic cells (DCs) play a key role in initiating and controlling the magnitude and the quality of adaptive immune responses^{1,2}. DCs decode and integrate such signals, and ferry this information to cells of the adaptive immune system. DCs are composed of subsets, which possess specialized as well as shared functions³⁻⁵. Microbes can directly activate DCs through a variety of pattern recognition receptors (PRR) such as Toll-like receptors (TLRs)⁶, cell surface C-type lectin receptors (CLRs)⁷, and intracytoplasmic NOD-like receptors (NLRB)^{8,9}. In humans, certain CLRs distinguish DC subsets, with plasmacytoid DCs (pDCs) expressing BDCA2¹⁰, Langerhans cells (LCs) expressing Langerin¹¹, and interstitial DCs expressing DC-SIGN¹². Other C-type lectins are expressed on other cell types including endothelial cells and neutrophils. CLRs, such as DC-SIGN⁷, can act as anchors for a large number of microbes and allow their internalization. Furthermore, CLRs also act as adhesion molecules between DCs and other cell types including endothelial cells, T cells, and neutrophils^{12,13}. DEC-205/CD205, a lectin of unknown function, has been extensively studied in the mouse for its ability to endocytose ligands. Targeting antigens to mouse DCs through DEC-205 in the absence of DC-activation results in tolerance induction^{14,15}. In contrast, targeting antigens in the presence of DC activation (CD40 and TLR3 agonists) results in the generation of immunity against a variety of antigens^{14,16}. Most studies demonstrating induction of CD4⁺ T cell responses or primary CD8⁺ T cell response against antigens delivered via DEC-205 has been limited to the transgenic mouse OT-I/II system.

Antigens have been targeted to mouse DCs via other surface molecules including LOX-1 (a type II C-type lectin receptor that binds to HSP70¹⁷), mannose receptor¹⁸, Dectin-1¹⁹, Dectin-2²⁰, CD40²¹, Langerin²², Gb3 (a receptor for Shiga toxin²³), DEC-205²⁴, and CLEC9A which was recently described to prime naïve CD8⁺ T cells in mice^{25,26,27}. The targeting of antigens through receptors expressed on different murine DC subsets results in different functional outcomes^{28,29}. Targeting antigens to human DCs Conjugates of anti-DC-SIGN with KLH³⁹, anti-DEC-205 with HIV gag³¹ and anti-mannose receptor with human chorionic gonadotropin hormone (hCGb)³² have been shown to be presented/crosspresented to blood CD4⁺ and CD8⁺ T cells, respectively, or to T cell clones.

The present inventors focus on lectin DCIR³³, which is widely expressed on different types DCs, including DCs from blood. Indeed, DCIR was initially described as expressed on blood monocytes, B cells, neutrophils, granulocytes and dermal DCs, but not LCs and was also recently found to be expressed on pDCs³⁴. Functionally it can serves as a receptor for HIV³⁵. The human genome encodes only a single DCIR gene while the mouse genome presents four DCIR-like genes DCIR2, DCIR3, DCIR4 and DCAR1. DCIR and DCAR share substantial sequence homology in their extracellular domains, However, DCAR associates with the immunoreceptor family tyrosine-based activation motif (ITAM)-bearing FcRγ chain, whereas, DCIR contains an immunoreceptor tyrosine-based inhibitory motif (ITIM) that recruits the SHP-1 and SHP-2 phosphatases³⁶. A human homolog for the mouse DCAR has not been identified thus far.

The instant invention reports the successful delivery of antigens to a wide range of DC subsets by an anti-DCIR conjugate mAb, allowing crosspresentation and crosspriming of human CD8⁺ T cells. Examples of DCIR-specific antibodies include (Accession #'s: PTA 10246 and PTA 10247, described previously in U.S. Patent Publication Nos. 20080241170 and 20080206262), relevant portions, including sequences, incorporated herein by reference).

DC subsets: CD34⁺-derived DCs were generated in vitro from CD34⁺-HPCs isolated from the blood of healthy volunteers given G-CSF to mobilize precursor cells. HPCs were cultured at 0.5×10⁶ cells/ml in Yssel's medium (Irvine Scientific, CA) supplemented with 5% autologous serum, 50 μM β-mercaptoethanol, 1% L-glutamine, 1% penicillin/streptomycin, GM-CSF (50 ng/ml; Berlex), Flt3-L (100 ng/ml; R&D), and TNF-α (10 ng/ml; R&D) for 9 days. Media and cytokines were refreshed at day 5 of culture. Subsets of DCs, CD1a′CD14⁻-LCs and CD1a⁻CD14⁺ DCs were then sorted, yielding a purity of 95-99%. Monocytes-derived DCs were generated by culturing monocytes in RPMI supplemented with 10% fetal bovine serum (FBS) with GM-CSF (100 ng/ml; Immunex Corp.) and IL-4 (25 ng/ml R&D) for 5 days, or with GM-CSF (100 ng/ml; Immunex Corp.) and IFN-α2b (500 U/ml; Intron A; Schering-Plough) for 3 days. mDCs and pDCs were sorted from fresh PBMCs as Lin⁻HLA-DR⁺ CD11c⁺ CD123⁻ and Lin⁻HLA-DR⁺ CD11c⁻CD123⁺, respectively.

Epidermal LCs, dermal CD1a⁺ DCs, and dermal CD14⁺ DCs were purified from normal human skin specimens. Specimens were incubated in bacterial protease dispase type 2 for 18 h at 4° C., and then for 2 h at 37° C. Epidermal and dermal sheets were then separated, cut into small pieces (−1-10 mm) and placed in RPMI 1640 supplemented with 10% FBS. After 2 days, the cells that migrated into the medium were collected and further enriched using a Ficoll-diatrizoate in a density of 1.077 g/dl. DCs were purified by cell sorting after staining with anti-CD1a FITC (DAKO) and anti-CD14 APC mAbs (Invitrogen). All protocols were reviewed and approved by the institutional review board.

Expansion of antigen-specific T cells in DC/T cell coculture: To assess the function of DCs in presenting FluMP- or MART-1-derived antigens, the present inventors used DCs from HLA-A201⁺ donors. Cells were cultured with conjugates mAbs at the indicated concentration. Syngeneic purified CD8⁺ T cells were cultured with the antigen-pulsed DCs at a DC/T ratio 1:20. CD40L (100 ng/ml; R&D) was added to the culture after 24 h to enhance crosspresentation by DCs⁴⁹. The cocultures were incubated at 37° C. for 8-10 days. IL-2 was added at 10 U/ml at day 3. Where indicated, DCs were activated with TLR agonists: LPS (10, 50 or 200 ng/ml; Invivogen), Poly I:C (5, 10 or 25 μg/ml) or Thiazoloquinoline compound CL075 (0.2, 1 or 2 μg/ml; Invivogen). The expansion of FluMP-, MART-1-, and HIV gag p24-specific CD8⁺ T cells was evaluated using HLA-A201-FluMP (58-66) peptide (GILGFVFTL) (SEQ ID NO: 1), HLA-A201-MART-1 (26-35) peptide (ELAGIGILTV) (SEQ ID NO: 2) and HLA-A201-p24 (151-155) peptide (TLNAWVKVV) (SEQ ID NO: 3)-tetramers, respectively (Beckman Coulter). For the assessment of crosspriming to multiple CD8⁺ T cell-specific epitopes, CD34⁺-derived DCs were incubated with anti-DCIR-p24 or IgG4-p24 fusion mAbs and cultured with CFSE-labeled CD8⁺ T cells at a DC/T ratio 1:30. Antigen-pulsed DCs were activated with CD40L (100 ng/ml). After two consecutive stimulations, the CFSE^{low} proliferating cells were sorted and restimulated for 24 h with fresh DCs loaded with HIV gag p24 protein (2 μg/ml). The secreted IFN-γ was measured in the culture supernatants by Luminex. Alternatively, mDCs or IFN-α DCs were targeted with anti-DCIR-MART-1 or IgG4-MART-1 fusion proteins, activated as indicated and cultured with naïve CD8⁺ T cells for 10 days. Production of intracellular IFN-γ, as well as mobilization of CD107a (BD Biosciences) where indicated, was measured after 5 h of restimulation with fresh autologous DCs that were loaded with 15 amino acid overlapping peptides derived from the MART-1 protein (2.5 μM) in the presence of the protein transport inhibitor monensin (GolgiStop; BD Biosciences). Secretion of IFN-γ, TNF-α, IL-12p40, IL-4, IL-5 and IL-13 were measured in the supernatant after 40 h by Luminex.

Additional methods of the instant invention include details presented herein below on the generation of anti-DCIR mAbs and production of recombinant DCIR, cloning and expression of chimeric mouse/human IgG4 recombinant mAbs, DCIR expression analysis on APCs, DCIR-signaling effect on the DC phenotype and function, cloning and production of fusion protein mAbs, peptide-MHC complexes detection on DCs, purification of CD8⁺ T cells and crosspresentation of FluMP protein by chemically-linked anti-DCIR mAb.

Generation of anti-DCIR mAbs and production of recombinant DCIR: Mouse mAbs were generated by conventional cell fusion technology. Briefly, 6-week-old BALB/c mice were immunized intraperitonealy with 20 μg of receptor ectodomain.hIgGFc fusion protein with Ribi adjuvant, then boosted with 20 μg antigen 10 days and 15 days later. After 3 months, the mice were boosted again three days prior to taking the spleens. Alternately, mice were injected in the footpad with 1-10 μg antigen in Ribi adjuvant every 3-4 days over a 30-40 days period. B cells from spleen or lymph node cells were fused with SP2/O—Ag 14 cells⁵¹ using conventional techniques. ELISA was used to screen hybridoma supernatants against the receptor ectodomain fusion protein compared to the fusion partner alone or versus the receptor ectodomain fused to AP³³. Positive wells were then screened by flow cytometry using HEK293F cells transiently transfected with expression plasmids encoding full-length receptor cDNA. Selected hybridomas were single cell cloned and expanded in CELLine flasks (Intergra). Hybridoma supernatants were mixed with an equal volume of 1.5 M glycine, 3 M NaCl, 1×PBS, pH 7.8 (binding buffer) and tumbled with MabSelect resin (eBiosciences) (800 μl/5 ml supernatant). The resin was washed and eluted with 0.1 M glycine, pH 2.7. Following neutralization with 2 M Tris, mAbs were dialyzed versus PBS. The anti-DCIR antibody AB8-26.9E8.1E3 (HS854), Deposit No. PTA-10246, was deposited with the American Type Culture Collection (ATCC) or anti-DCIR antibody Deposit No. PTA 10247.

cDNA cloning and expression of chimeric mouse/human recombinant IgG4 mAbs. Total RNA was prepared from hybridoma cells (RNeasy kit, Qiagen) and used for cDNA synthesis and PCR (SMART RACE kit, BD Biosciences) using supplied 5′ primers and gene-specific 3′ primers mIgGκ (5′ggatggtgggaagatggatacagttggtgcagcatc3′) (SEQ ID NO: 4) and mIgG1 (5′gtcactggctcagggaaatagcccttgaccaggcatc3′) (SEQ ID NO: 5). PCR products were then cloned (pCR2.1 TA kit, Invitrogen) and characterized by DNA sequencing. Using the derived sequences for the mouse heavy (H) and light (L) chain variable (V) region cDNAs, specific primers were used to PCR amplify the signal peptide and V-regions, while incorporating flanking restriction sites for cloning into expression vectors encoding downstream human IgG1 (or IgG4H regions. The vector for expression of chimeric mVκ-hIgGκ was built by amplifying residues 401-731 (gi|63101937|) flanked by Xho I and Not I sites and inserting this into the Xho I-Not I interval of the vector pIRE7A-DsRed2 (BD Biosciences). PCR was used to amplify the mAb Vκ region from the initiator codon, appending a Nhe I or Spe I site then CACC, to the region encoding (e.g., residue 126 of gi|76779294|), appending a Xho I site. The PCR fragment was then cloned into the Nhe I-Not I interval of the above vector. The control hIgG4H vector corresponds to residues 12-1473 of gi|19684072| with 7A29P and L236E substitutions, which stabilize a disulphide bond and abrogate residual FcR interaction³⁸, inserted between the Bgl II and Not I sites of pIRE7A-DsRed2 (BD Biosciences) while adding the sequence 5′-gctagctgattaattaa-3′(SEQ ID NO: 6) instead of the stop codon. PCR was used to amplify the mAb VH region from the initiator codon, appending CACC then a Bgl II site, to the region encoding residue 473 of gi|19684072|. The PCR fragment was then cloned into the Bgl II-Apa I interval of the above vector. The vector for chimeric mVH-hIgG4 sequence using the mSLAM leader was built by inserting the sequence 5′ctagttgctggctaatggaccccaaaggctccctttcctggagaatacttctgtttctctccctggcttttgagttgtcgtacggattaattaagggc cc3′(SEQ ID NO: 7) into the Nhe I-Apa I interval of the above vector. PCR was used to amplify the interval between the predicted mature N-terminal codon and the end of the mVH region while appending 5′tcgtacgga3′. The fragment digested with Bsi WI and Apa I was inserted into the corresponding sites of the above vector. Antigen coding sequences flanked by a proximal Nhe I site and a distal Not I site following the stop codon were inserted into the Nhe I-Pac I-Not I interval of each H chain vector. Dockerin (Doc) was encoded by gi|40671| C. thermocellum CelD residues 1923-2150 with proximal Nhe I site and a distal Not I site. HIV gag p24 was encoded by gi|77416878| residues 133-363 with a proximal Nhe I site and sequence from gi|125489020| residues 60-75 and a distal Not I site. Recombinant antibodies were produced using the FreeStyle™ 293 or CHO—S Expression Systems (Invitrogen) according to the manufacturer's protocol (1 mg total plasmid DNA with 1.3 ml 293 Fectin reagent or 1 mg total plasmid DNA with 1 ml FREESTYLE MAX reagent/L of transfection, respectively). Equal amounts of vectors encoding the H and L chain were co-transfected. Transfected cells were cultured for 3 days, then the culture supernatant was harvested and fresh media with 0.5% penicillin/streptomycin (Biosource) added with continued incubation for 2 days. The pooled supernatants were clarified by filtration, loaded onto a 1 ml HiTrap MabSelect™ column, eluted with 0.1 M glycine pH 2.7, neutralized with 2 mM Tris and then dialyzed versus PBS with Ca⁺⁺/Mg⁺⁺. Proteins were quantified by absorbance at 280 nm.

DCIR expression analysis: DCIR expression was assessed on PBMCs, in vitro generated- or skin-derived DCs. Cells were double stained with anti-DCIR mAb (generated as described in supplemental methods), or mouse IgG1 (BD), washed, and then stained with PE-conjugated goat anti-mouse IgG (BD Pharmingen), then washed and incubated with FITC or APC-conjugated anti-CD3, anti-CD19, anti-CD11c, anti-HLA-DR, anti-CD11c, anti-CD123, anti-CD56, anti-CD16, (BD Pharmingen) anti-CD1a (DAKO) or anti-CD14 (Invitrogen) mAbs. Epidermal sheets were stained as detailed in supplementary methods to assess DCIR expression on immature LCs.

For the expression of DCIR on immature LCs, epidermal sheets were cut into approximately 10 mm squares and placed in 4% paraformaldehyde for 30 min. Sheets were washed in PBS and blocked with Background Buster (Innovex) for 30 min. Epidermal sheets were then incubated overnight with 0.5 μg purified mouse anti-DCIR (clone 9E8) or control IgG1, washed twice with PBS/0.05% Saponin and incubated for 1 h with a secondary goat anti mouse IgG-Alexa568 (Molecular Probes) (1:500 dilution). Nuclei were stained with DAPI (Invitrogen; Molecular Probes) at 1:5000 followed by 2 h incubation with anti-HLA-DR-FITC. Sheets were rinsed with PBS and mounted in Vectamount (Vector Laboratories). All antibodies were diluted in CytoQ diluent and block (Innovex) and all incubations were at 4° C. with constant mild agitation. Images were taken with an Olympus Planapo 20/0.7, Coolsnap HQ camera and analyzed using Metamorph software.

DCIR-signaling effect on DC-function: CD34⁺-derived DCs were cultured in anti-DCIR (clone 24A5 or 9E8) or isotype control coated plates in the presence or absence of CD40L (R&D; 100 ng/ml) or LPS (Invivogen; 50 ng/ml). After 24 h, cells were harvested and stained for surface phenotype. The secreted cytokines were analyzed by a multiplex bead assay (Luminex). For a global gene signature analysis 0.5×10⁶ epidermal cells that were purified from normal human skin were exposed to either anti-DCIR (clone 24A5 or 9E8), anti-CD40 (clone 12E12) or an IgG1 isotype matched control in a soluble, cross-linked or plate coated form at 5 μg/ml for 24 h. Double-stranded cDNA was obtained from 200 ng of total RNA and after in vitro transcription underwent amplification and labeling steps according to the manufacturer's instructions. 1.5 μg of amplified biotin-labeled cRNA was hybridized to the Illumina Sentrix Hu6 BeadChips according to the sample labeling procedure recommended by Illumina (Ambion, Inc, Austin, Tex.). BeadChips consist of 50mer oligonucleotide probes attached to 3-μm beads within microwells on the surface of the glass slide representing 48,687 probes. Slides were scanned on Illumina BeadStation 500 and Beadstudio software was used to assess fluorescent hybridization signals. To study the effect of DCIR signaling on allogeneic CD8⁺ T cell priming, LCs were cultured with allogeneic naïve CD8⁺ T cells in a plate coated with anti-DCIR mAb or IgG1 control (10 μg/ml) at ratio DC:T 1:20 in the presence or absence of CD40L. T cell proliferation responses were assayed by measuring [³H]-thymidine incorporation during the last 12 h of 6 days cultures. The proliferating CD8⁺ T cells (CFSE^{low}) were analyzed for their phenotype and their cytokine secretion pattern following CD3/CD28 mAb stimulation. To study the effect of DCIR signaling on autologous CD8⁺ T cells priming, CD34⁺-derived DC subsets were loaded with the HLA-A201-restricted MART-1 (26-35) peptide and cocultured with naïve CD8⁺ T cells in the presence of a soluble form of anti-DCIR mAb or IgG1 control (10 μg/ml) and CD40L. After 10 days, cells were harvested and analyzed for the frequency of MART-1-specific CD8⁺ T cells by specific tetramer, and for the expression of effector molecules Granzyme A (BD Pharmingen), Granzyme B (eBiosciences) and perforin (Fitzgerald).

Cloning and production of fusion protein mAbs: FluMP was chemically cross-linked to mAbs using sulfosuccinimidyl 6-[3′(2-pyridyldithio)-propionamido]hexanoate (sulfo-LC-SPDP; Pierce) according to the manufacturer's protocol. Chimeric mouse/human recombinant mAbs anti-DCIR and control IgG4 were fused to a ˜9.5 kDA dockerin domain in-frame with the rAb H chain. The entire FluMP, containing the immuno-dominant HLA-A201-restricted FluMP (58-66) peptide (GILGFVFTL) (SEQ ID NO: 1), and a sequence encoding the immuno-dominant HLA-A201-restricted MART-1 (26-35) peptide (ELAGIGILTV) (SEQ ID NO: 2) from the melanoma MART-1 antigen with surrounding natural MART-1 residues: DTTEARHPHPPVTTPTTDRKGTTAEELAGIGILTVILGGKRTNNSTPTKGEFCRYPSHWRP (SEQ ID NO: 8), were each fused to the ˜17.5 kDa cohesin domain and were expressed in E. coli strains BL21 (DE3) (Novagen) or T7 Express (NEB). Recombinant mAb (rAb)-antigen conjugate was formed by mixing rAb.Doc fusion protein with 2 molar equivalents of cohesin.antigen fusion protein. The dockerin and cohesin domains self-associate to form a stable [rAb.doc-coh.antigen] conjugate (as described in Flamar et al.). The chimeric rAb anti-DCIR or IgG4 control antibodies were fused to the HIV gag p24 protein⁵² or to a portion of a recombinant form of the MART-1 protein. The anti-DCIR-MART-1 (clone 9E8) fusion protein used had the following peptide units appended to the H chain C-terminus [each unit flanked by AS residues]: Bacteroides cellulosolvens cellulosomal anchoring scaffoldin B precursor [gb|AAT79550.1|] residues 651-677 with a T672N substitution; MART-1|gb|BC014423.1| residues 1-38; gb|AAT79550.1| residues 1175-1199; MART-1 residues 78-118. For cell-surface staining of mAb-FluMP conjugates, coh.FluMP was biotinilated using EZ-Link NHS-SS-PEO₄-Biotin (Pierce) according to the manufacturer's procedure. Monocyte-derived DCs were stained with 10 μg/ml rAb.doc-coh.FluMP.Biotin complexes on ice for 20 min. Cell-surface binding was detected using PE-conjugated Streptavidin (1:200; BD Biosciences) and analyzed by flow cytometry.

Peptide-MHC complexes detection on DCs: CD34⁺-derived DCs from an HLA-A201⁺ donor were incubated with 50 nM DCIR.doc-coh.FluMP conjugate or free coh.FluMP fusion protein in culture media supplemented with 10% human serum, 50 ng/ml GM-CSF and 10 ng/ml TNF-α. 5 μg/ml anti-CD40 mAb (12E12, BIIR) was added after 2 h. Cells were assessed after 24 h for FluMP (58-66) peptide (GILGFVFTL)-HLA-A201 complexes by flow cytometry using PE-conjugated tetramerized M1D12 monoclonal antibody⁵³.

Purification of CD8⁺ T cells: CD8⁺ T cells were negatively selected from PBMCs using CD14, CD19, CD16, CD56 and CD4 magnetic beads, or purified using the naïve CD8⁺ T cell isolation kit (Miltenyi Biotec). In some experiments, naive CD8⁺ T cells were sorted as CD8⁺CCR7⁺CD45RA⁺ and memory CD8⁺ T cells were sorted as CD8⁺ CCRTCD45RA⁺. Where indicated, cells were labeled with 5 μM carboxyfluorescein diacetate succinimidyl ester (CFSE; Invitrogen).

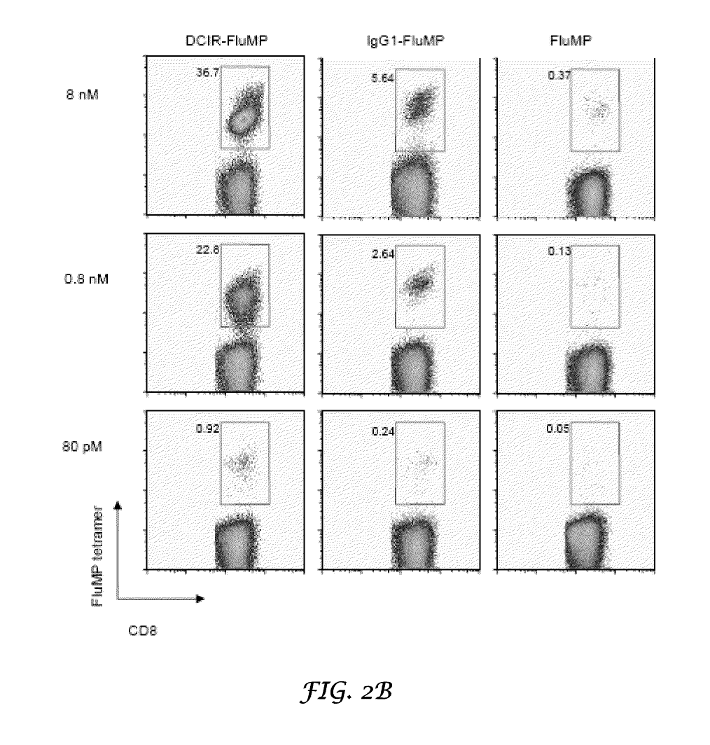

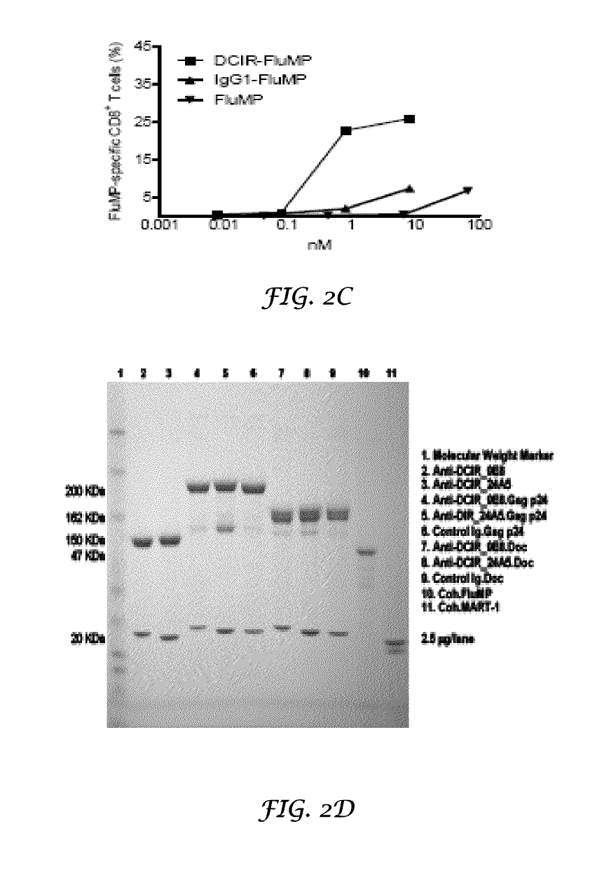

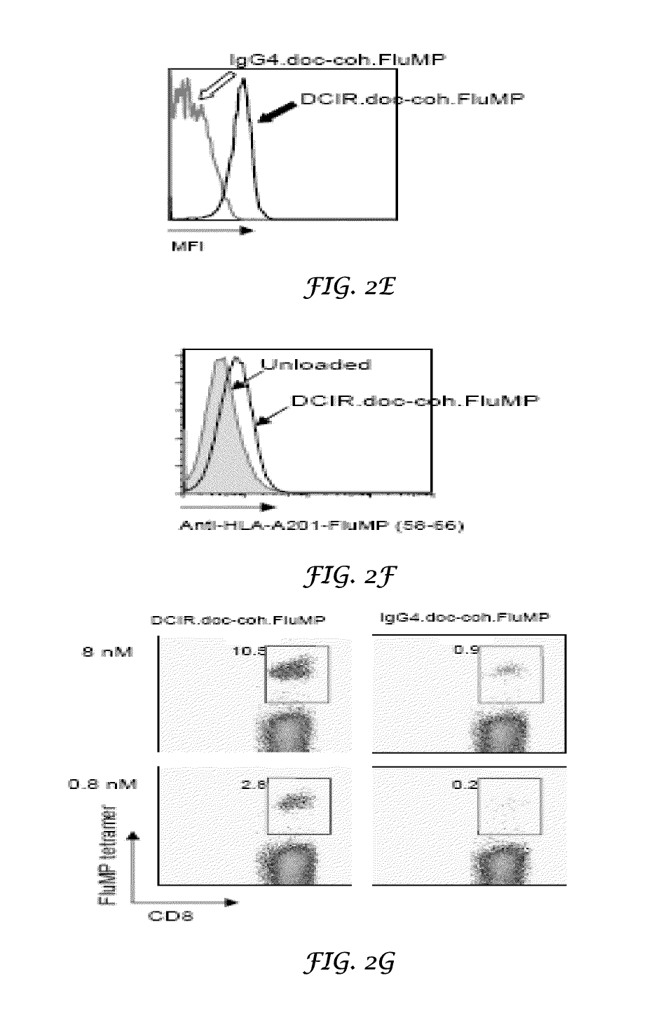

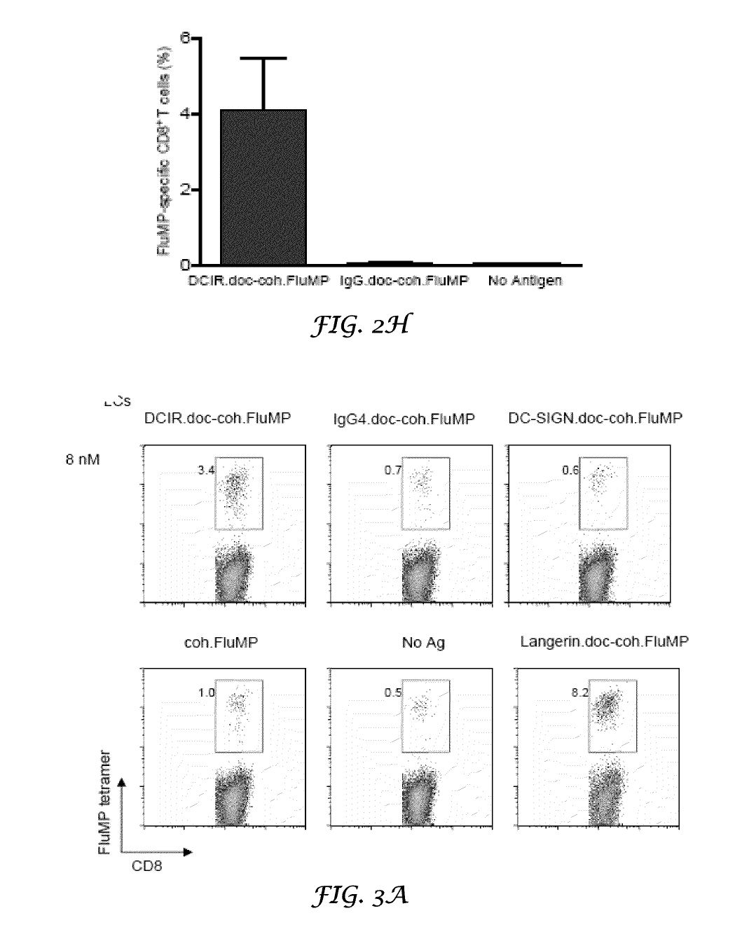

Crosspresentation of FluMP protein by chemically-linked anti-DCIR mAb: CD34⁺-derived LCs from an HLA-A201⁺ donor were cultured for 8 days with purified CD8⁺ T cells together with increasing concentrations of either anti-DCIR-FluMP or controls including IgG1-FluMP and free FluMP protein. When delivered alone, FluMP induced very limited expansion of FluMP-specific CD8⁺ T cells (FIG. 2B) as assessed by staining with a FluMP (58-66)-specific HLA-A201-tetramer. IgG1-FluMP was more efficient than the free FluMP protein, suggesting Fc-mediated uptake⁵⁴. Dose-titration curve, illustrated in FIG. 2C, shows that anti-DCIR-FluMP elicited a response with at least 50-fold less antigen than the control IgG1-FluMP or the free FluMP, therefore demonstrating actual targeting of the antigen. Note that the free antigen never induced the high frequency of FluMP-specific CD8⁺ T cells observed with anti-DCIR-FluMP (FIG. 2C).

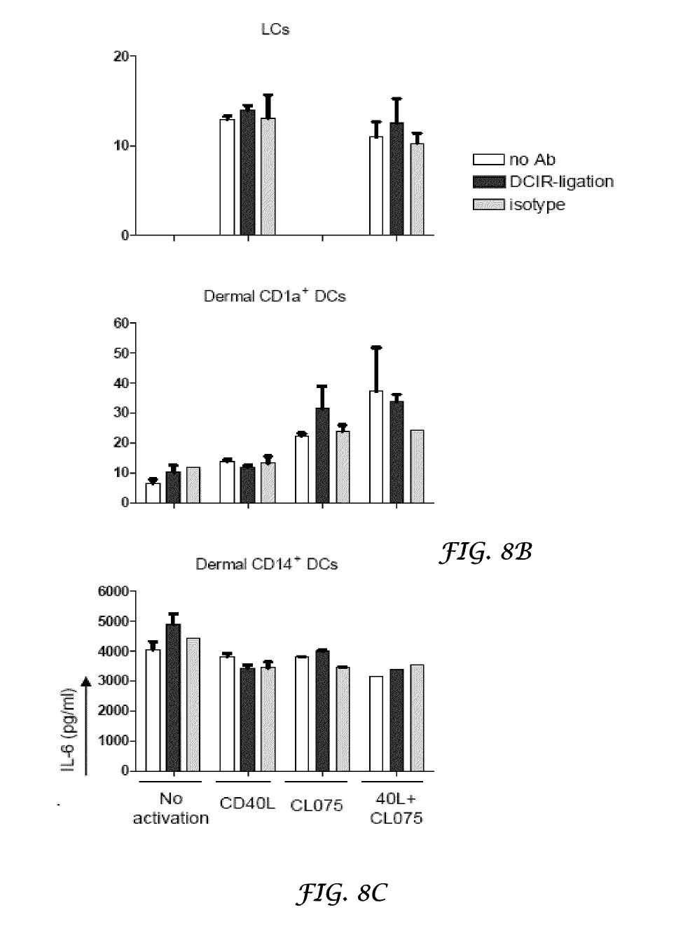

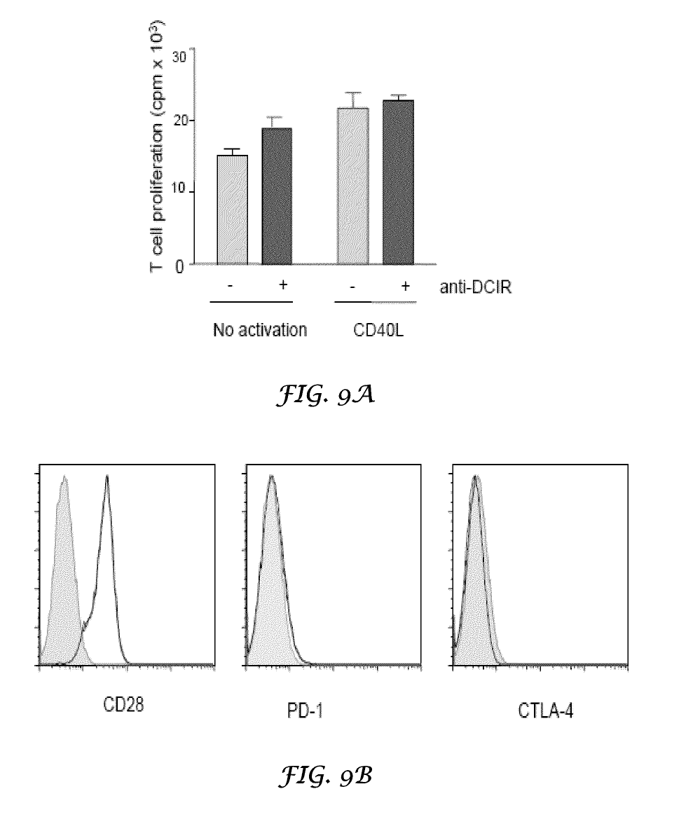

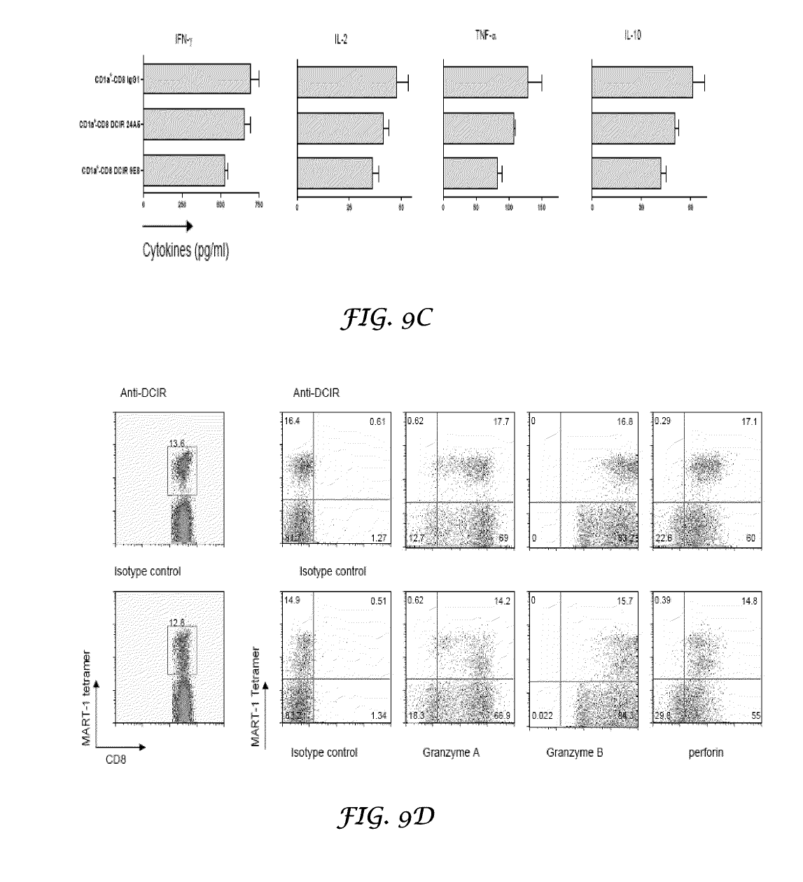

Table 1 indicates the mean fluorescence expression of CD80, CD86,CD40, ICOS-L, HLA-ABC and HLA-DR on the surface of CD1a⁺ LCs that were stimulated for 24 h with anti-DCIR or isotype control in the presence or absence of CD40L. FIG. 2B shows the proportions of HLA-A201-FluMP (58-66) peptide tetramer-positive CD8⁺ T cells expanded by CD1a⁺ LCs cultured with cross-linked anti-DCIR-FluMP, crosslinked control IgG-FluMP proteins, or free FluMP. FIG. 2C shows the percentage of FluMP-specific CD8⁺ T cells in response to decreasing concentrations of cross-linked mAb-FluMP constructs or free FluMP. FIG. 2D shows SDS-PAGE-reducing gel of mouse and chimeric anti-DCIR mAbs, as well as protein antigens used in this study. FIG. 2E shows binding of the anti-DCIR.doc-coh.FluMP conjugate mAb to the surface of monocyte-derived DCs. FIG. 8A shows flow cytometry analysis of the expression of CD86 on the surface of CD1a⁺ LCs (S3A) and IL-6 secretion by Luminex by skin isolated DCs (LCs, dermal CD1a⁺ DCs and dermal CD14⁺ DCs) (FIG. 8B) that were stimulated for 24 h with anti-DCIR or isotype control in the presence or absence of CD40L. Data shows that the anti-DCIR antibody did not alter the phenotype of cultured DCs, nor did it inhibit CD40- or CL075-induced activation. FIG. 8C shows that only CD40-ligation but not DCIR ligation induced global activation gene signature by epidermal skin cells that were exposed to a soluble, cross-linked or plate coated form of the mAb. FIG. 9A shows that anti-DCIR antibodies did not alter the proliferation of naïve T cells elicited by allogeneic CD1a⁺ LCs. FIGS. 9B and 9C show that addition of anti-DCIR antibodies did not alter the phenotype (PD-1, CTLA-4, and CD28) and cytokine secretion (IFN-γ, IL-2, TNF-α and IL-10) of naïve CD8⁺ CD45RA⁺ T cells activated by allogeneic DCs. FIG. 9D shows that anti-DCIR antibody did not alter the ability of MART-1 peptide-loaded DCs to prime MART-1-specific effector CD8⁺ T cells as analyzed by flow cytometry with a specific teteramer and by the level of effector molecules (Granzyme A, Granzyme B and perforin).

TABLE 1
Mean fluorescence expression of CD80, CD86, CD40, ICOS-L,
HLA-ABC and HLA-DR on the surface of CD1a⁺ LCs
that were stimulated for 24 h with anti-DCIR or isotype
control in the presence or absence of CD40L.

| | CD80 | CD86 | CD40 | HLA-ABC | ICOS-L | HLA-DR |
|---|---|---|---|---|---|---|
| no intent | 161.0 | 76.6 | 8.9 | 62.1 | 17.200 | 501.000 |
| +DCIR | 139.0 | 87.0 | 10.5 | 61.2 | 21.800 | 424.000 |
| C040L | 241.0 | 294.0 | 31.8 | 108.0 | 69.800 | 1018.000 |
| CD40L + | 235.0 | 282.0 | 27.1 | 100.0 | 73.600 | 809.000 |
| DCIR | | | | | | |

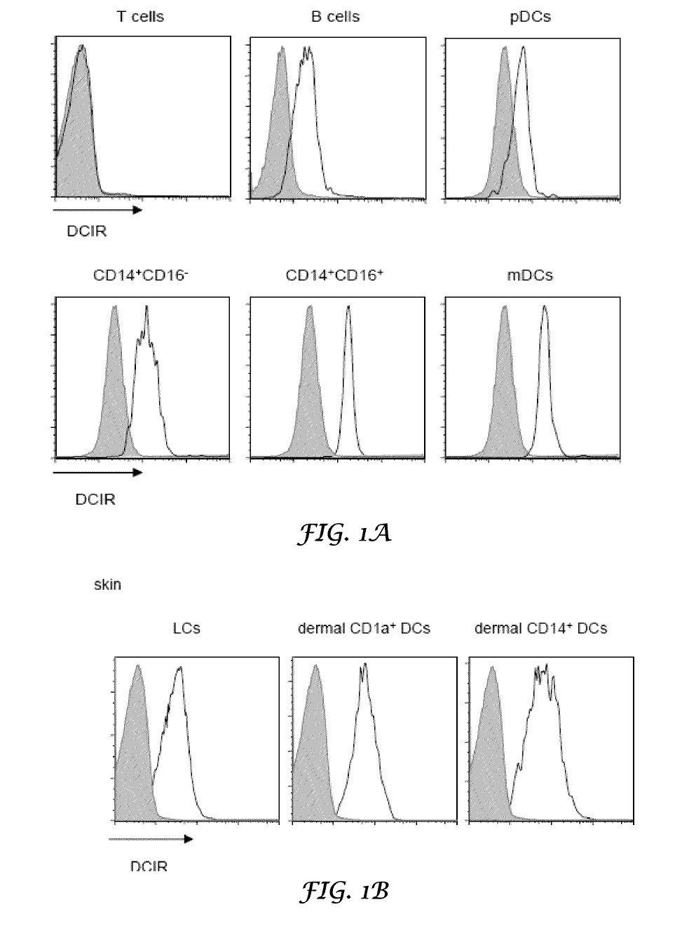

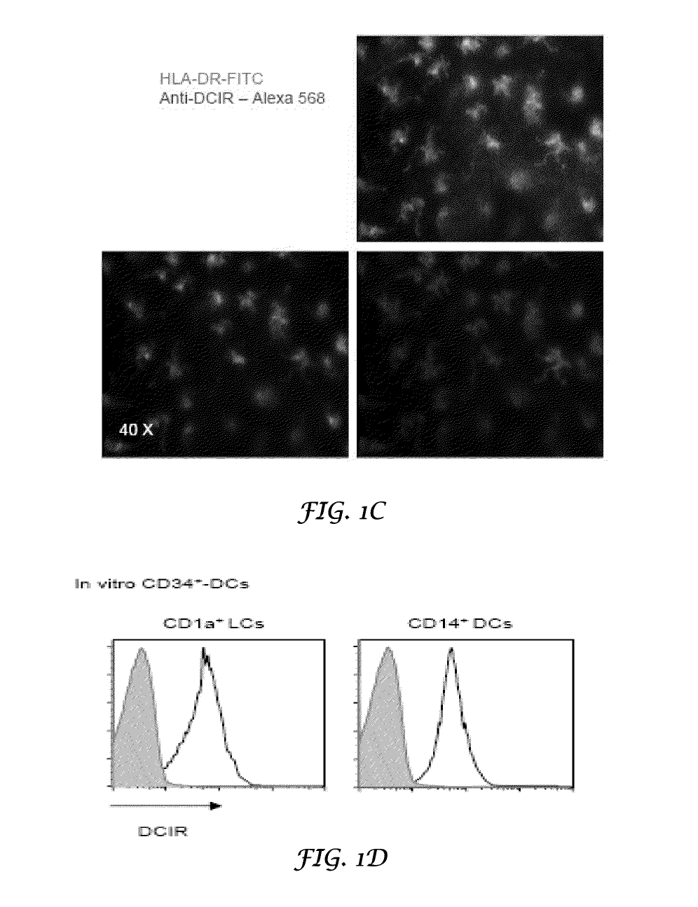

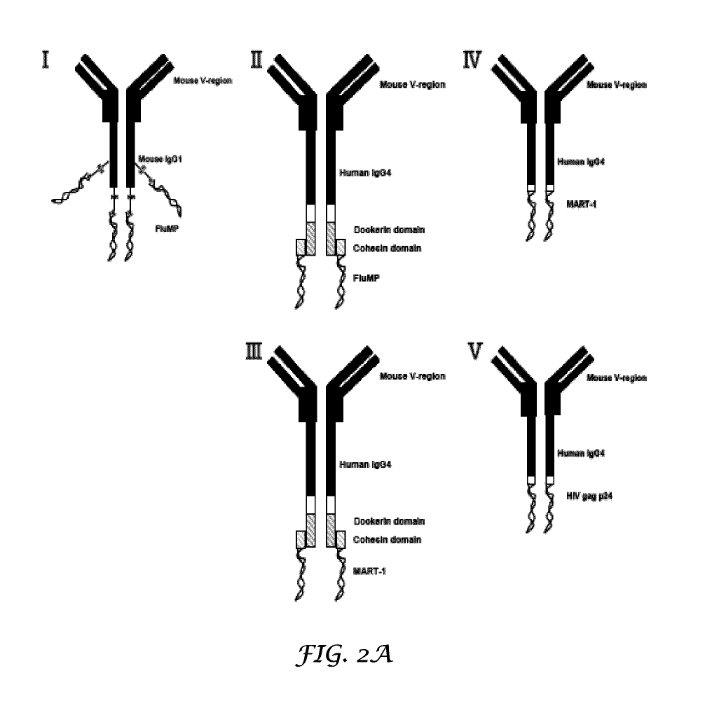

DCIR is expressed by monocytes, B cells and all DC subsets: Two monoclonal anti-DCIR clones were used throughout the studies: 9E8 and 24A5. These proved to be of high affinity (˜850 pM and ˜560 pM, respectively) as assessed by surface plasmon resonance analysis. They showed comparable staining of PBMCs (FIG. 1A) and yielded comparable functional results throughout the present study.

DCIR was found to be expressed by all circulating APCs as indicated by HLA-DR expression. These APCs include the CD14⁺ monocytes (both CD14⁺CD16⁻ and CD14⁺CD16⁺ subsets), LIN⁻HLA-DR⁺CD11c⁺ blood myeloid DCs (mDCs), LIN⁻HLA-DR⁺CD11c⁻CD123⁺ plasmacytoid DCs (pDCs) and on CD19⁺ B lymphocytes. DCIR was not detected on CD3⁺ T cells (FIG. 1A) or CD16⁺ and CD56⁺ NK cells (not shown). DCIR was expressed on purified epidermal LCs, dermal CD14⁻CD1a⁺, and dermal CD14⁺CD1a⁻DCs (FIG. 1B). Immunofluorescence analysis of epidermal sheets further confirmed the expression of DCIR on HLA-DR⁺ LCs in situ (FIG. 1C). DCIR is expressed on CD1a⁺ LCs and CD14⁺ interstitial DCs generated in vitro by culturing CD34⁺ hematopoietic progenitor cells (HPCs) with a combination of GM-CSF, FLT3-L and TNF-α for nine days³⁷ (FIG. 1D), as well as on monocyte-derived DCs cultured with GM-CSF and IL-4, or with GM-CSF and type I IFN (not shown).

Thus, the findings of the present invention confirm the earlier findings of DCIR expression on monocytes, B cells, dermal DCs, mDCs and pDCs^{33,34}, and further show its expression on skin LCs.

Crosspresentation of FluMP protein by anti-DCIR conjugates: Studies using a FluMP protein chemically coupled to anti-DCIR antibody (FIG. 2A, Construct I) demonstrated that when linked to an antigen, DCIR allows crosspresentation of the immunodominant HLA-A201-restricted FluMP (58-66) peptide (FIGS. 2B and 2C). This led us to construct fusion proteins based on recombinant anti-DCIR (or control IgG4 antibodies) and FluMP, but these failed to be efficiently secreted from transfected HEK293F cells. We therefore designed a strategy based on the high affinity interaction (˜30 pM) between cohesin and dockerin, two proteins of the cellulosome from Clostridium thermocellum (Flamar et al.). The mAb.Dockerin fusion protein (mAb.doc) (FIGS. 2A construct II and 2D) was readily secreted by transfected mammalian cells and purified on a protein A affinity column. The control hIgG4H and recombinant anti-DCIR antibodies each carry S229P and L236E substitutions, which stabilize a disulphide bond and abrogate residual FcR interaction³⁸. FluMP was produced in E. coli as a soluble Cohesin fusion protein (coh.FluMP) (FIGS. 2A construct II and 2D). Targeting conjugates were generated by incubating equimolar amounts of mAb.doc and coh.FluMP for 15 minutes before being delivered to DCs. The recombinant anti-DCIR.doc-coh.FluMP complex mAb (full arrow) bound to the surface of human monocyte-derived DCs, while the control conjugate IgG4-FluMP (empty arrow) did not bind the cells (FIG. 2E).

To determine whether the recombinant anti-DCIR.doc-coh.FluMP complex mAb was processed and presented by DCs, DCs from an HLA-A201⁺ donor were cultured for 24 h with 50 nM conjugate mAb and stained with the monoclonal antibody (M1D12) that detects FluMP (58-66) peptide bound to HLA-A201. DCs exposed to anti-DCIR-FluMP conjugate mAb display HLA-A201-FluMP (58-66) peptide complexes on their surface (black histogram) (FIG. 2F).

To assess presentation of antigen to purified CD8⁺ T cells, the recombinant conjugate mAbs were offered at two concentrations (8 nM and 0.8 nM) to CD34⁺-HPC-derived LCs. Anti-DCIR.doc-coh.FluMP, at 8 nM, was more potent in inducing the expansion of FluMP-specific CD8⁺ T cells than the IgG4.doc-coh.FluMP (10.5% tetramer positive cells vs. 0.9%) (FIG. 2G, upper panel). The potency of targeting via DCIR was confirmed at a lower conjugate mAb concentration (0.8 nM) where the control conjugate mAb was barely crosspresented (2.8% vs. 0.2% positive cells) (FIG. 2G, lower panel). The ability of DCIR to target antigen to DCs was further illustrated when the DCs were exposed for only 18 h to the conjugate mAbs (8 nM) and washed before culturing with CD8⁺ T cells (4.12±2.13% vs. 0.05±0.02% tetramer positive cells) (FIG. 2H).

Thus targeted delivery of antigen to ex vivo-generated DCs via DCIR allows efficient crosspresentation of proteins to CD8⁺ T cells.

Anti-DCIR conjugates allow crosspresentation of proteins by skin Langerhans cells blood mDCs and blood pDCs: Inasmuch as these fusion proteins are intended to be used as vaccines, we assessed whether the constructs would be crosspresented by human DC subsets isolated from either skin or blood. Thus, 8 nM of the recombinant anti-DCIR.doc-coh.FluMP complex was added to cultures of 5×10³ sorted epidermal HLA-A201⁺ LCs and 1×10⁵ purified blood CD8⁺ T cells for 10 days (FIG. 3). This resulted in expansion of FluMP-specific CD8⁺ T cells by DCIR-targeted LCs when compared to the IgG4 control complex mAb (3.4% vs. 0.7%). Free coh.FluMP (1%) or a complex against a lectin which is not expressed by LCs, i.e. DC-SIGN.doc-coh.FluMP (0.6%) (FIG. 3A) were very weakly crosspresented if at all. An antibody antigen complex against Langerin, a Langerhans cells-specific lectin induced expansion of FluMP-specific CD8⁺ T cells by LCs (8.2%). The expansion of tetramer-specific CD8⁺ T cells (FIG. 3A) correlated with the levels of IFN-γ measured in the culture supernatant (FIG. 3B).

Both subsets of blood DCs, CD11c⁺ mDCs and BDCA2⁺ pDCs, express DCIR (FIG. 1A). Thus, mDCs and pDCs purified from the same cytapheresis samples³⁹ were tested for their ability to crosspresent FluMP delivered via DCIR. 5×10³ DCs were cultured with 1×10⁵ autologous CD8⁺ T cells and decreasing concentrations of either free coh.FluMP, or IgG4.doc-coh.FluMP conjugate or the anti-DCIR coh.FluMP conjugate.

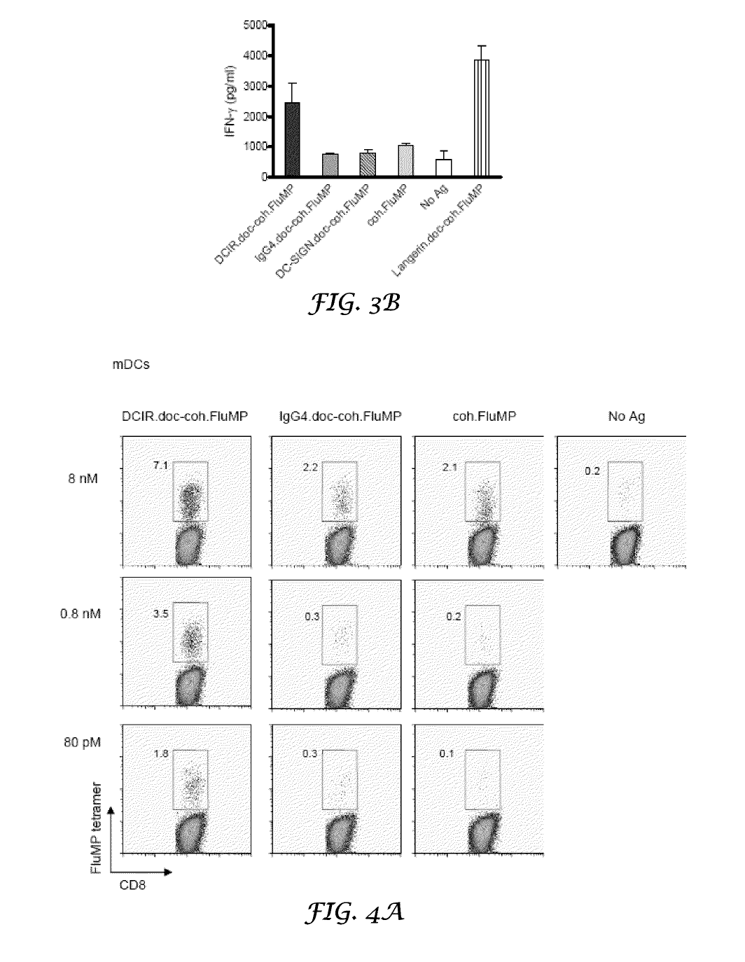

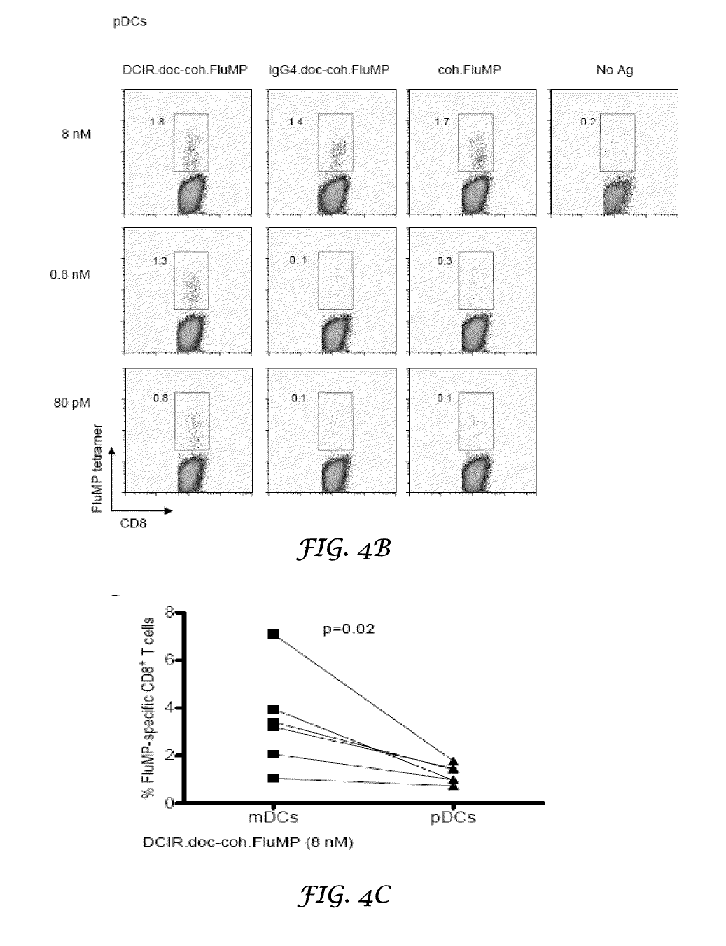

The anti-DCIR.doc-coh.FluMP complex mAb (FIG. 4A) efficiently targeted FluMP to mDCs since concentrations as low as 80 pM yielded 1.8% tetramer positive cells. Coh.FluMP itself and the control IgG4.doc-coh.FluMP conjugate were able to induce expansion of antigen-specific CD8⁺ T cells only at 8 nM.

pDCs were also able to crosspresent the three forms of recombinant FluMP at a concentration of 8 nM. At 0.8 nM and 80 pM, anti-DCIR.doc-coh.FluMP complex mAb allowed crosspresentation of the FluMP antigen, while free coh.FluMP, or IgG4.doc-coh.FluMP conjugates were not crosspresented (FIG. 4B). When compared to pDCs, mDCs targeted with 8 nM of anti-DCIR.doc-coh.FluMP complex mAb were able to induce a more robust expansion of FluMP-specific CD8⁺ T cells (as measured with a specific HLA-A201 tetramer) (FIG. 4C) (p=0.02).

Taken together, these data indicate that anti-DCIR mAb potently targets proteins for crosspresentation by skin Langerhans cells, blood mDCs and pDCs.

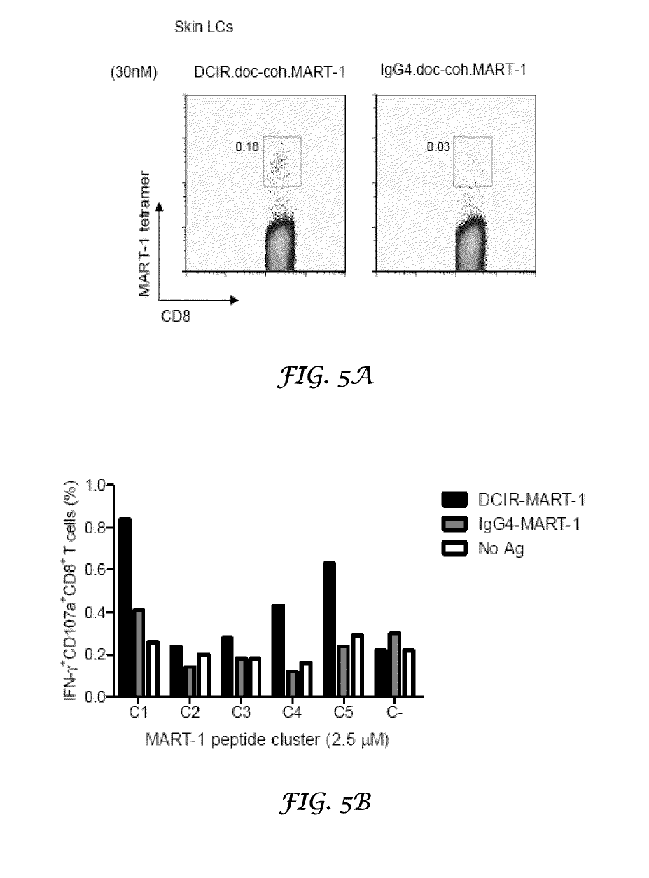

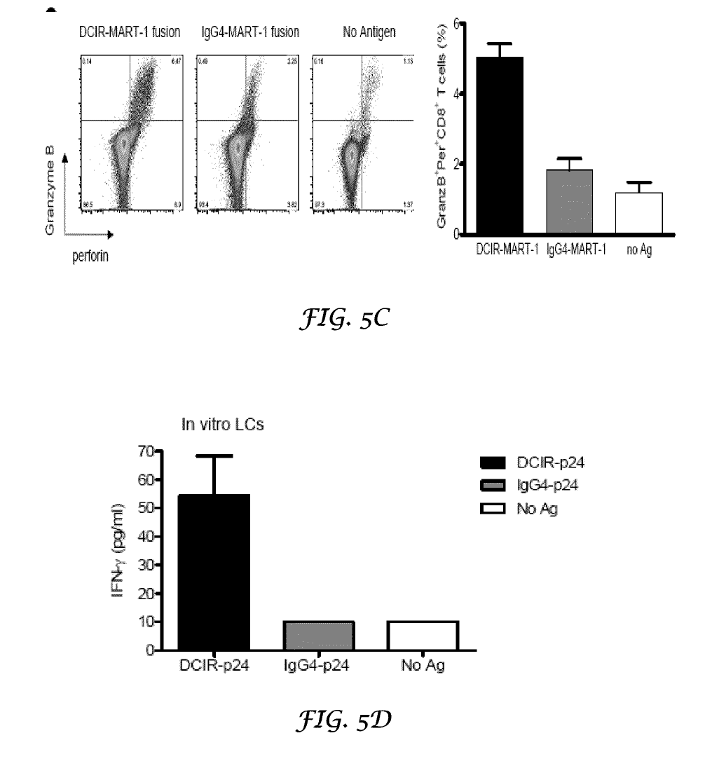

Crosspriming of MART-1 and HIV gag proteins by anti-DCIR conjugates: The present inventors further tested whether DCIR would permit the crosspriming of naïve CD8⁺ T cells using: i) anti-DCIR.dockerin and cohesin fused to the 10-mer MART-1 (26-35) HLA-A201-restricted peptide (EAAGIGILTV) (FIG. 2A, III) (SEQ ID NO: 9); ii) anti-DCIR directly fused to MART-1 recombinant protein (FIG. 2A, IV) or to HIV gag p24 protein (FIG. 2A, V). Epidermal HLA-A201⁺ LCs were cultured with autologous T cells with 30 nM anti-DCIR.doc-coh.MART-1 or IgG4.doc-coh.MART-1 complex mAbs. After 10 days, the binding of MART-1 (26-35)-HLA-A201⁺ tetramer indicated that anti-DCIR.doc-coh.MART-1 complex mAb allowed skin-derived LCs to prime CD8⁺ T cells and expand MART-1-specific CD8⁺ T cells (FIG. 5A).

The successful expression of anti-DCIR-MART-1 fusion protein (FIG. 2A, IV) allowed us to further assess crosspriming to other epitopes of the MART-1 protein. Thus, DCs were exposed to either anti-DCIR-MART-1 or IgG4-MART-1 fusion protein or to no protein, activated with CD40L and cultured with autologous purified naïve CD8⁺ T cells. After 10 days, cells were re-stimulated for 5 h with DCs loaded with clusters of individual peptides derived from the MART-1 protein or with unloaded DCs. Mobilization of CD107a, a marker for cytotoxic activity determination, to the cell surface and the expression of intracytoplasmic IFN-γ were measured to assess specific CTL responses. Anti-DCIR-MART-1 fusion protein induced expansion of MART-1-specific CD8⁺ T cells to peptides from cluster 1, cluster 4 and cluster 5 of the MART-1 protein (FIG. 5B). Targeting DCs with DCIR-MART-1 fusion protein induced expansion of CD8⁺ T cells expressing high levels of the effector molecules Granzyme B and perforin (FIG. 5C).

Anti-DCIR-p24 and IgG4-p24 fusion proteins (FIG. 2A, V) were also well secreted form HEK293F cells. Thus, purified naïve CD8⁺ T cells from healthy individuals were labeled with CFSE and primed by two consecutive 7 day cultures with DCs and with either of these fusion proteins, or no protein. The proliferating CFSE^{low}CD8⁺ T cells were sorted and re-challenged with HIV gag p24 (p24) protein-loaded DCs. CD8⁺ T cells primed with anti-DCIR-p24 fusion protein (black bar) were able to secrete IFN-γ in response to the p24 challenge while control fusion proteins did not (grey bar) (FIG. 5D). This indicates specific priming of naïve CD8⁺ T cells by the anti-DCIR-p24 fusion protein.

The findings of the studies of the present invention demonstrate that targeting antigens via DCIR allows priming of CD8⁺ T cells specific for both self and non-self antigens.

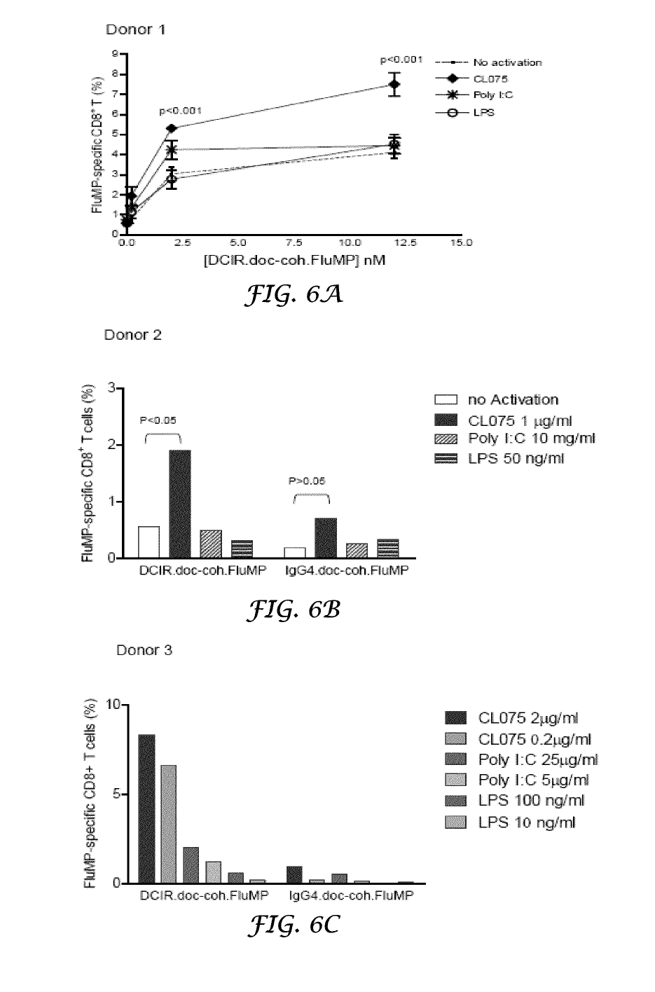

TLR7/8-agonist enhances DCIR-mediated crosspresentation: As TLR triggering activates DCs, we analyzed whether TLR ligands would enhance the antigen-specific CD8⁺ T cell responses induced by mDCs targeted with anti-DCIR complexes. 5×10³ purified blood HLA-A201⁺ mDCs were cultured with increasing amounts of anti-DCIR.doc-coh.FluMP complex mAb and agonists for TLR3 (Poly I:C; 5 μg/ml), TLR4 (LPS; 50 ng/ml) or TLR7/8 (CL075; 1 μg/ml) and 1×10⁵ autologous purified CD8⁺ T cells. The specific-FluMP CD8⁺ T cell response was measured after 8-10 days using HLA-A201-FluMP (58-66) tetramer. The TLR3-agonist (Poly I:C) enhanced the FluMP-specific responses at low concentration of the targeting complex (2 nM and 0.2 nM) while activation via TLR4 did not. The TLR7/8-agonist (CL075) was found to be the most potent in expanding FluMP-specific CD8⁺ T cells (FIG. 6A). The CL075-enhanced response was observed for all tested concentrations of anti-DCIR.doc-coh.FluMP complex and was dependent on the presence of the mAb targeting complex (FIGS. 6A and 6B). Increasing the concentrations of Poly I:C from 5 μg/ml to 25 μg/ml or LPS from 50 ng/ml to 200 ng/ml did not significantly enhance the expansion of the antigen-specific CD8⁺ T cells in response to the DCIR.doc-coh.FluMP complex mAb. TLR3-activation, however, resulted in higher FluMP-specific response than TLR4-activation (FIG. 6C). Low concentration of 0.2 μg/ml of the TLR7/8-agonist was sufficient to enhance the FluMP-specific response (FIG. 6C). No significant synergistic effect was seen when soluble CD40L was added in addition to the TLR-agonist (not shown).

For every tested concentration or combination of activators tested, FluMP-specific responses to anti-DCIR.doc-coh.FluMP were always significantly higher than those induced by the control IgG4.doc-coh.FluMP (FIGS. 6B and 6C), or free coh.FluMP (not shown). Thus, TLR7/8 activation enhances DCIR-dependent crosspresentation of protein antigen by mDCs.

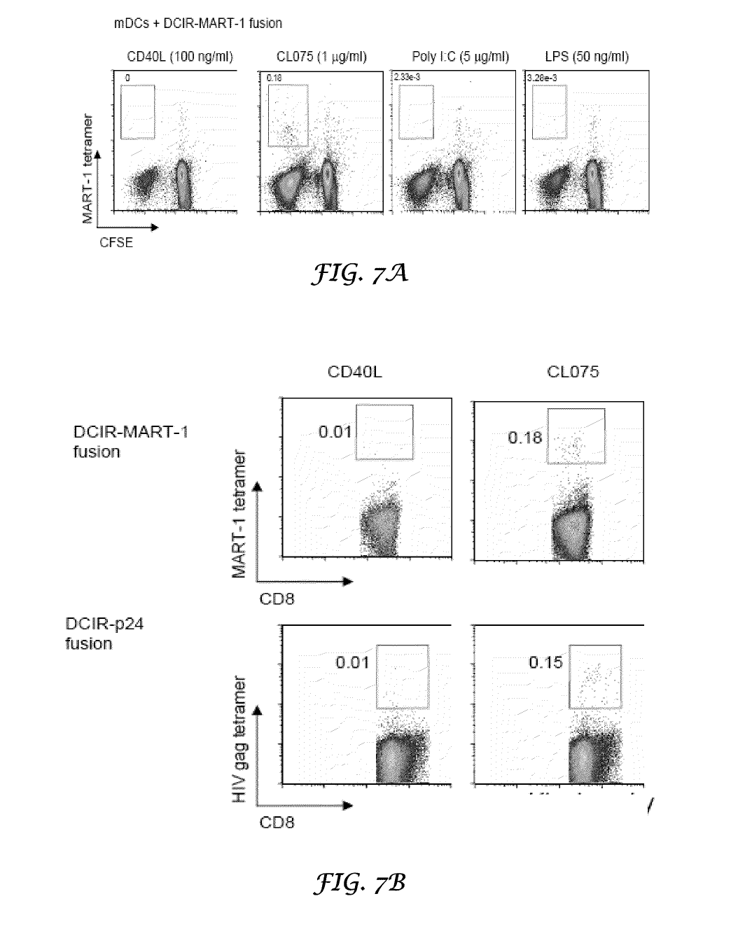

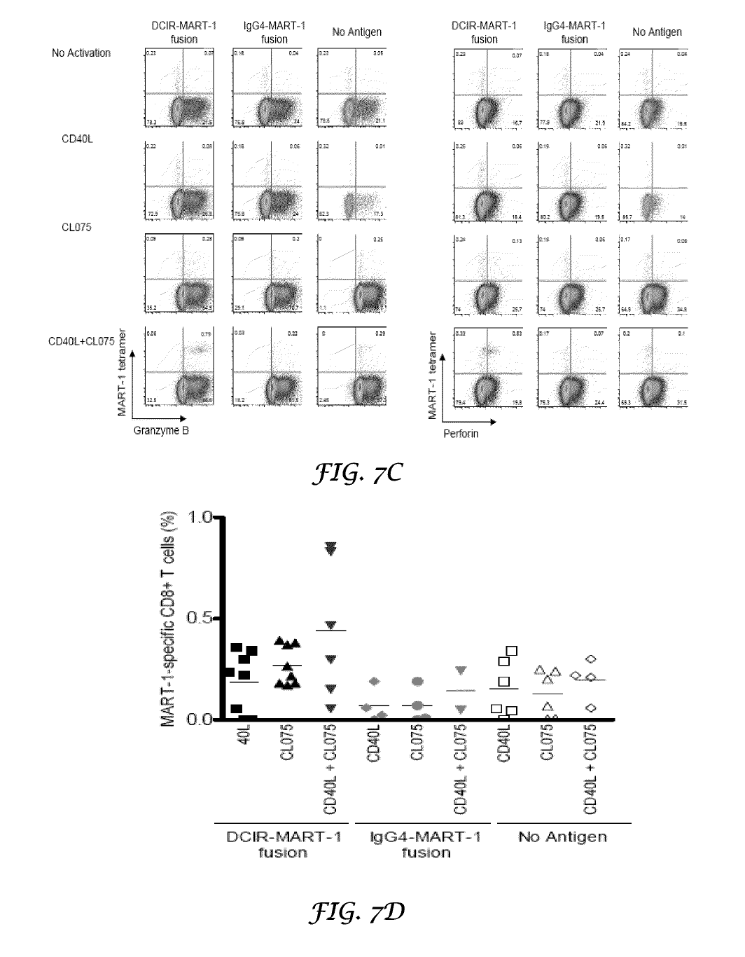

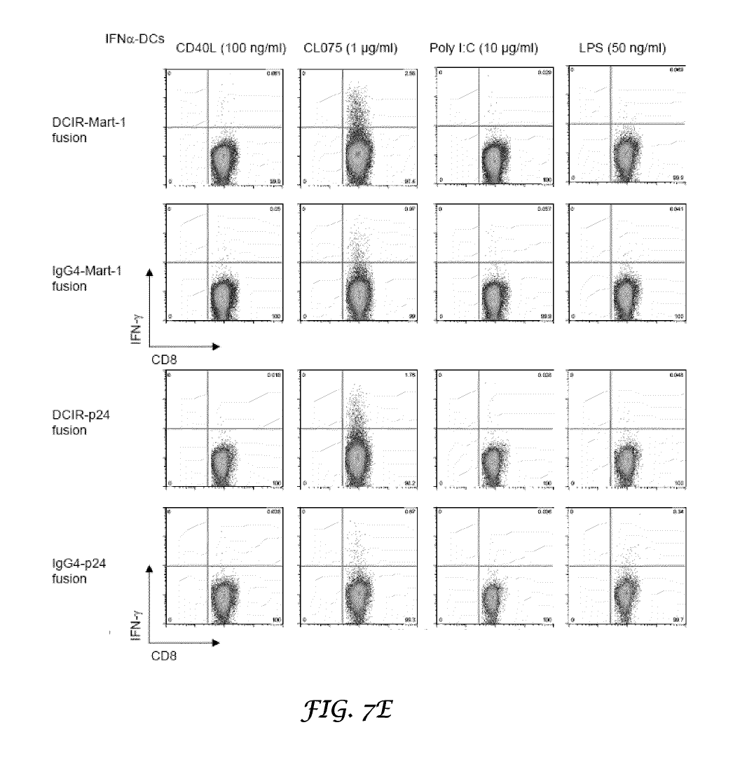

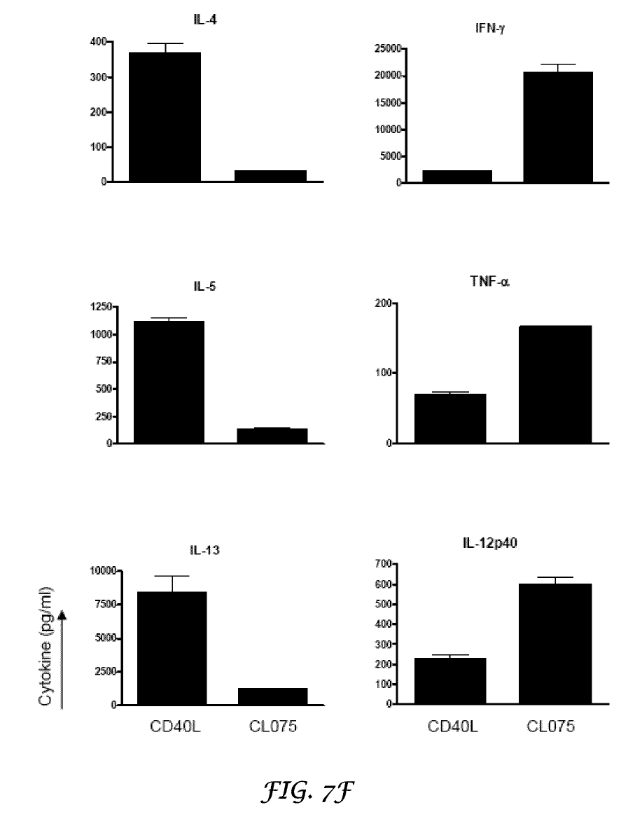

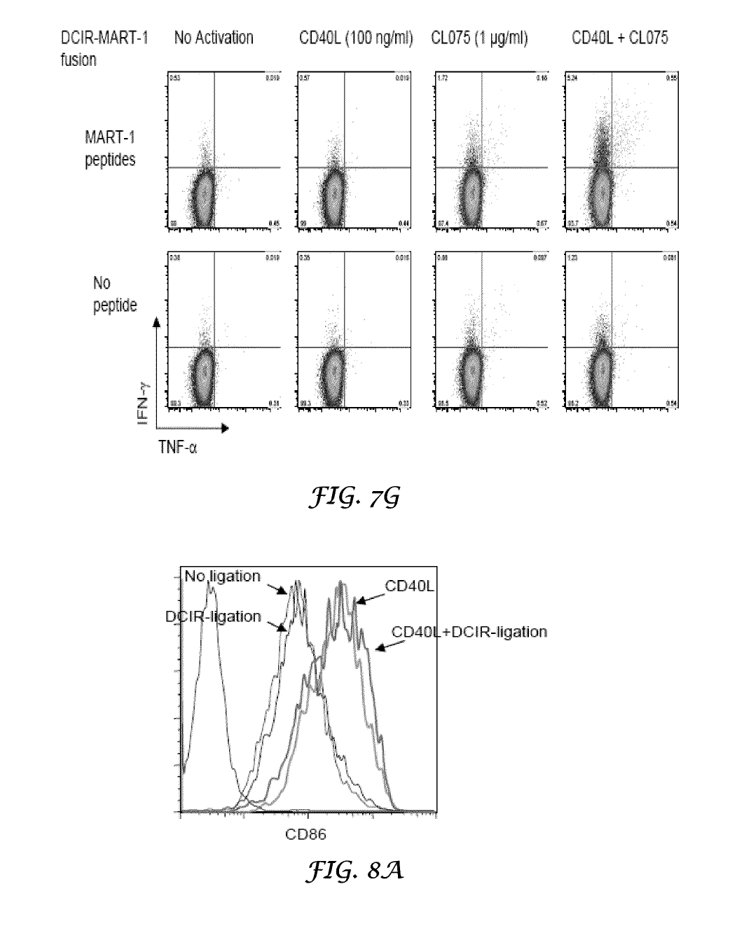

TLR7/8-agonist enhances DCIR-mediated crosspriming: The inventors further examined whether TLR7/8-ligand would also enhance DCIR-mediated primary CD8⁺ T cell responses. Blood HLA-A201⁺ mDCs were cultured with either anti-DCIR-MART-1 or the IgG4-MART-1 fusion protein (FIG. 2A, construct IV). The DCs were activated with CD40L, TLR3-L, TLR4-L or TLR7/8-L and cocultured with purified CFSE-labeled naïve CD8⁺ T cells. The expansion of MART-1 (26-35)-HLA-A2-restricted CFSE^{low}CD8⁺ T cells was assessed after 10 days using a specific tetramer (FIG. 7A). TLR7/8-activated DCs induced the highest expansion of MART-1-specific CD8⁺ T cells (0.18%) (FIG. 7A). In a second experiment using blood mDCs and a single dose of both anti-DCIR-MART-1 or anti-DCIR-p24 fusion protein (FIG. 2A, constructs IV and V) together with the TLR7/8-agonist, but not CD40L, induced expansion of MART-1 and HIV gag p24-HLA-A201-tetramer binding CD8⁺ T cells (0.18% vs. 0.01% and 0.15% vs. 0.01%) (FIG. 7B). Unlike secondary responses however, co-signaling via both CD40- and TLR7/8 resulted in a synergistic effect and a larger expansion of tetramer-binding CD8⁺ T cells compared to CD40L or TLR7/8-agonist alone (0.3% vs. 0.37% vs. 0.83%) (FIGS. 7C and 7D). Thus, TLR7/8-agonist enhances crosspriming and crosspresentation of antigen-specific CD8⁺ T cells.

TLR7/8-ligand increases CTL effector molecules and decrease type 2 cytokine production: The next set of studies was designed to determine whether TLR7/8-triggering during DCIR-targeting would alter the quality of the elicited responses. Thus naïve CD8⁺ T cells were cultured with autologous HLA-A201⁺ mDCs and anti-DCIR-MART-1 fusion protein without activation or with CD40L or CL075 alone, or CD40L+CL075. After 10 days, cells were stained with HLA-A201-MART-1 (26-35) tetramer and Granzyme B or perforin-specific mAbs. Compared to each activator alone, the combination of CD40L and TLR7/8 agonist induced higher expression of the effector molecules Granzyme B (FIG. 7C; left panel) and perforin (FIG. 7C; right panel) by the expanded CD8⁺ T cells.

Compared to CD40L-, Poly I:C- or LPS-conditioned DCs, CD8⁺ T cells that were primed by DC targeted with anti-DCIR-MART-1 fusion protein and TLR7/8-agonist, expressed higher amounts of IFN-γ in response to a specific-restimulation with autologous DCs loaded with peptides from the MART-1 protein (FIG. 7E; upper panel). A second model antigen, HIV gag p24, allowed us to further demonstrate the effect of TLR7/8-ligand on the quality of the primed CD8⁺ T cells. Thus, CD8⁺ T cells primed by anti-DCIR-p24 fusion protein-targeted DCs and activated with TLR7/8-agonist, expressed higher amounts of IFN-γ compared to CD40L-, Poly I:C- or LPS-activated DCs in response to a specific restimulation with autologous DCs loaded with 15 amino acid-overlapping peptides from the HIV gag p24 protein (FIG. 7E; lower panel). As expected, the level of intracytoplasmic IFN-γ was higher when the antigen was delivered via DCIR compared to a control IgG4 mAb (FIG. 7E). Interestingly, DCIR-primed CD8⁺ T cells produced a different set of cytokines, in response to reactivation with MART-1 peptide-loaded DCs, according to whether they were initially exposed to either CD40L- or CL075-triggered DCs. (FIG. 7F). While CD40L-matured IFN-α DCs induced naïve CD8⁺ T cells to express high amounts of type 2 cytokines (IL-4, IL-5 and IL-13), TLR7/8-exposed DCs educated naïve CD8⁺ T cells to preferentially secrete IFN-γ and TNF-α with markedly reduced amounts of IL-4, IL-5 and IL-13 (FIG. 7F). Furthermore, compared to each activator alone, a combination of TLR7/8 and CD40L induced the most robust expansion of IFN-γ and TNF-α-producing CD8⁺ T cells in response to a restimulation with 15 amino acid-overlapping peptides derived from the MART-1 protein, as observed by intracellular staining (FIG. 7G). Thus, TLR7/8-activation alters the quality of primary CD8⁺ T cell responses by DCIR-targeted mDCs, by enhancing IFN-γ secretion and reducing type 2 cytokine secretion.

Studies presented hereinabove were initiated on the premise that ligation of DCIR, a surface lectin that expresses an ITIM motif, will result in deactivation or prevention of activation of DCs. As described earlier, DCIR is expressed at high density on blood monocytes and at lower levels on B cells³³. DCIR is also expressed at high density on purified dermal CD14⁺ DCs in accordance with earlier immunohistochemistry data³³. However, at variance with these data, DCIR was found to be expressed on epidermal Langerhans cells, after their purification, as well as on intact epidermal sheets. The discrepancy of the two studies regarding LCs is intriguing as DCIR expression is also observed with LCs generated in vitro by culturing CD34⁺ HPC with GM-CSF and TNF-α³⁷. We, and others, also found DCIR to be expressed at high density on blood myeloid DCs⁴⁰ and blood plasmacytoid DCs³⁴. Thus, DCIR is expressed by all human DC subsets of blood and skin DCs.

Engaging DCIR with 12 different anti-DCIR antibodies neither inhibited nor enhanced DC activation as measured by either expression of CD80, CD83 and CD86 or the secretion of cytokine (such as IL-6, IL-12). DCIR cross-linking neither enhanced nor inhibited the DC-mediated proliferation of CD4⁺ and CD8⁺ T cells. In addition, as assessed by microarray analysis, ligation of DCIR, as opposed to CD40, did not reveal an activation gene signature by isolated epidermal cells (data not shown). However, evidence for the inhibitory role of DCIR has been documented in dcir-deficient mice that showed an exacerbated response to collagen-induced arthritis, with increased numbers of activated DCs and activated CD4⁺ T cells⁴¹. It should however be noted that mouse and human differ considerably at the level of the DCIR gene complex inasmuch as the mouse genome encodes four DCIR-like molecules: DCIR-2, DCIR-3, DCIR-4 and DCAR-1, while the human genome encodes a single one. Alternative explanations include the possibility that the mAbs we have generated are unable to provide negative signals, or that our antibodies crossreact with an as yet unidentified human counterpart of the mouse activating receptor DCAR. Another possibility might be that the inhibitory signal of DCIR is delivered in cells other than DCs, i.e., monocytes or B cells³⁶. In the human, a recent study³⁴ demonstrated a slight inhibition of TLR9-induced IFN-γ production by pDCs, without affecting the expression of co-stimulatory molecules, and reduced IL-12 and TNF-α production by TLR8-activated mDCs⁴⁰. Finally, as demonstrated for other lectins such as BDCA-2¹⁰ and DCAL-2⁴², depending on the cellular context, ITIMs can sometimes stimulate rather than repress cellular activation⁴³.

Antigens delivered through the receptor DCIR were found to be efficiently crosspresented to memory T cells. A concentration of anti-DCIR.doc-coh.FluMP complex mAb as low as 80 pM was sufficient to induce significant expansion of FluMP-specific CD8⁺ T cells. This represents an approximately 100-fold enhancement of the intrinsic antigen presentation capacity. Such an effect has been earlier reported in murine studies with fusion proteins of DEC-205¹⁶. A remarkable finding is that all the tested DC subsets were found to be targeted by the DCIR fusion proteins and induce a specific CD8⁺ T cell response. Indeed, in variance with previous studies^{33,34}, anti-DCIR was able to efficiently deliver antigens to blood pDCs as well as epidermal Langerhans cells and allowed development of specific CD8⁺ T cell responses. Antigen delivery through DCIR not only allowed the expansion of memory FluMP-specific CD8⁺ T cells, but also resulted in the priming of naïve CD8⁺ T against the melanoma differentiation antigen MART-1 and the HIV gag p24 protein. Furthermore, DCIR mediated response was broad and specific to multiple epitopes of MART-1 protein. Recently a monoclonal antibody to DCIR2 was found to preferentially target the CD8-DCIR2⁺ subset in mice, resulting in preferential induction of MHC Class II-restricted reactivation of CD4⁺ T cells²⁸. Likewise anti-DCIR was shown to target KLH to human pDCs thereby allowing proliferation of a KLH-specific CD4⁺ T cell line³⁴. The present invention demonstrates that DCIR is also a powerful means to establish and reactivate antigen-specific CD8⁺ T cell responses. All DCs including skin Langerhans cells, blood mDCs and pDCs were efficient at crosspresenting antigen delivered through DCIR. All together these data indicate that antigen delivery through DCIR, like DEC-205, can result in the induction of both MHC Class I and MHC Class II restricted immune responses.

Inasmuch as future vaccines will likely be composed of these targeted antigens together with an adjuvant, we have also addressed whether microbial (TLR) stimulation would improve DCIR-mediated antigen crosspresentation by mDCs. Among all the tested activators, TLR7/8 agonist proved most effective in this process and induced the highest proliferation of antigen-specific effector CD8⁺ T cells in both primary and secondary responses, particularly in the case of primary responses, when is delivered together with a CD40 signal. In addition to amplifying the specific CD8⁺ T cell response, TLR7/8-triggering also affected the quality of the induced T cells by promoting high expression of IFN-γ, and effector molecules such as Granzyme A, Granzyme B and perforin. Moreover, while DCIR-targeted IFN-α DCs activated with CD40L-primed CD8⁺ T cells to produce high amounts of Type 2 (IL-4, IL-5, and IL-13) cytokines, TLR7/8-agonist shifted the balance towards a Type 1-response, which is associated with enhanced production of proinflammatory cytokines IFN-γ and TNF-α and markedly reduced levels of IL-4, IL-5, and IL-13. Our findings are in accordance with previous observations attributing enhanced protein-based vaccine induced-T cell responses to TLR7/8-triggering^{44,45}. In a non human primate model of SIV, a protein antigen delivered along with a TLR7/8-ligand promoted the induction of a Th1 response, as well as the enhanced and durable expansion of multi-functional CD8⁺ T cells. These cells, which simultaneously produce IFN-γ, TNF-α, and IL-2 are abundant in HIV nonprogressor relative to progressors and associated with long term protection. Therefore, combining TLR7/8-agonist with a targeted protein-based vaccine should be beneficial to treat chronic diseases in which CD8⁺ T cells are mediating effector functions.

In the settings of the present invention, the possibility that the TLR agonists we used had also a direct effect on the CD8⁺ T cells cannot be excluded. As some studies previously demonstrated, direct TLR-triggering on CD4⁺ T cells can induce upregulation of costimulatory molecules and modulate their proliferation^{46,47}. Nevertheless, it has been demonstrated that the most effective multi-functional CD8⁺ T cell response is induced when the antigen is fused to the adjuvant, rather than delivered separately⁴⁴, a finding which might explain the lack of CD8⁺ T cell responses in melanoma patients vaccinated with NY-ESO and topical TLR7 agonist⁴⁸. Thus, our own data support the approach of conjugating TLR-agonists to a targeting antigen vaccine, such as DCIR, as the most efficient method to deliver an antigen and adjuvant directly to DCs. More studies are however required to formally conclude which activator or a combination of activators, and which vaccine formulation will yield the most potent, long lasting CD8⁺ T cell responses in vivo.

In summary, targeting clinically relevant antigens through DCIR to various DC subsets will permit induction of strong cytotoxic CD8⁺ T cell responses which are essential for the prevention and treatment of chronic diseases.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,387,271: Immunostimulatory Combinations.
U.S. Patent Publication No. 20080267984: Activation of Human Antigen-Presenting Cells through Dendritic Cell Lectin-Like Oxidized LDL Receptor-1 (LOX-1).
U.S. Patent Publication No. 20080241170: Vaccines Based on Targeting Antigen to DCIR Expressed on Antigen-Presenting Cells.
U.S. Patent Publication No. 20080241139: Adjuvant Combinations Comprising A Microbial TLR Agonist, A CD40 or 4-IBB Agonist, and Optionally An Antigen and the Use Thereof for Inducing A Synergistic Enhancement in Cellular Immunity.
U.S. Patent Publication No. 20100135994: HIV Vaccine Based on Targeting Maximized GAG and NEF to Dendritic Cells.
U.S. Patent Publication No. 20110081343: Vaccines Directed to Langerhans Cells.
U.S. Patent Publication No. 20080241170: Vaccines Based on Targeting Antigen to DCIR Expressed on Antigen-Presenting Cells.
U.S. Patent Publication No. 20080206262: Agents that Engage Antigen-Presenting Cells through Dendritic Cell Asialoglycoprotein Receptor (DC-ASGPR).
1. Banchereau J, Steinman R M. Dendritic cells and the control of immunity. Nature. 1998; 392:245-252.
2. Steinman R M, Banchereau J. Taking dendritic cells into medicine. Nature. 2007; 449:419-426.
3. Ueno H, Klechevsky E, Morita R, et al. Dendritic cell subsets in health and disease. Immunol Rev. 2007; 219:118-142.
4. Shortman K, Naik S H. Steady-state and inflammatory dendritic-cell development. Nat Rev Immunol. 2007; 7:19-30.
5. Klechevsky E, Morita R, Liu M, et al. Functional specializations of human epidermal langerhans cells and CD14+ dermal dendritic cells. Immunity. 2008; 29:497-510.
6. Takeda K, Kaisho T, Akira S. Toll-like receptors. Annu Rev Immunol. 2003; 21:335-376.
7. Geijtenbeek T B, van Vliet S J, Engering A, t Hart B A, van Kooyk Y. Self- and nonself-recognition by C-type lectins on dendritic cells. Annu Rev Immunol. 2004; 22:33-54.
8. Ye Z, Ting J P. NLR, the nucleotide-binding domain leucine-rich repeat containing gene family. Curr Opin Immunol. 2008; 20:3-9.
9. Meylan E, Tschopp J, Karin M. Intracellular pattern recognition receptors in the host response. Nature. 2006; 442:39-44.
10. Dzionek A, Sohma Y, Nagafune J, et al. BDCA-2, a novel plasmacytoid dendritic cell-specific type II C-type lectin, mediates antigen capture and is a potent inhibitor of interferon alpha/beta induction. J Exp Med. 2001; 194:1823-1834.
11. Valladeau J, Ravel O, Dezutter-Dambuyant C, et al. Langerin, a novel C-type lectin specific to Langerhans cells, is an endocytic receptor that induces the formation of Birbeck granules. Immunity. 2000; 12:71-81.
12. Geijtenbeek T B, Torensma R, van Vliet S J, et al. Identification of DC-SIGN, a novel dendritic cell-specific ICAM-3 receptor that supports primary immune responses. Cell. 2000; 100:575-585.
13. van Gisbergen K P, Sanchez-Hernandez M, Geijtenbeek T B, van Kooyk Y. Neutrophils mediate immune modulation of dendritic cells through glycosylation-dependent interactions between Mac-1 and DC-SIGN. J Exp Med. 2005; 201:1281-1292.
14. Hawiger D, Inaba K, Dorsett Y, et al. Dendritic cells induce peripheral T cell unresponsiveness under steady state conditions in vivo. J Exp Med. 2001; 194:769-779.
15. Kretschmer K, Apostolou I, Hawiger D, Khazaie K, Nussenzweig M C, von Boehmer H. Inducing and expanding regulatory T cell populations by foreign antigen. Nat. Immunol. 2005; 6:1219-1227.
16. Bonifaz L C, Bonnyay D P, Charalambous A, et al. In Vivo Targeting of Antigens to Maturing Dendritic Cells via the DEC-205 Receptor Improves T Cell Vaccination. J Exp Med. 2004; 199:815-824.
17. Delneste Y, Magistrelli G, Gauchat J, et al. Involvement of LOX-1 in dendritic cell-mediated antigen cross-presentation. Immunity. 2002; 17:353-362.
18. Tan M C, Mommaas A M, Drijfhout J W, et al. Mannose receptor-mediated uptake of antigens strongly enhances HLA class II-restricted antigen presentation by cultured dendritic cells. Eur J. Immunol. 1997; 27:2426-2435.
19. Carter R W, Thompson C, Reid D M, Wong S Y, Tough D F. Preferential induction of CD4+ T cell responses through in vivo targeting of antigen to dendritic cell-associated C-type lectin-1. J. Immunol. 2006; 177:2276-2284.
20. Carter R W, Thompson C, Reid D M, Wong S Y, Tough D F. Induction of CD8+ T cell responses through targeting of antigen to Dectin-2. Cell Immunol. 2006; 239:87-91.
21. Schjetne K W, Fredriksen A B, Bogen B. Delivery of antigen to CD40 induces protective immune responses against tumors. J. Immunol. 2007; 178:4169-4176.
22. Idoyaga J, Cheong C, Suda K, et al. Cutting Edge: Langerin/CD207 Receptor on Dendritic Cells Mediates Efficient Antigen Presentation on MHC I and II Products In Vivo. J. Immunol. 2008; 180:3647-3650.
23. Vingert B, Adotevi O, Patin D, et al. The Shiga toxin B-subunit targets antigen in vivo to dendritic cells and elicits anti-tumor immunity. Eur J. Immunol. 2006; 36:1124-1135.
24. Bozzacco L, Trumpfheller C, Huang Y, et al. HIV gag protein is efficiently cross-presented when targeted with an antibody towards the DEC-205 receptor in Flt3 ligand-mobilized murine DC. Eur J Immunol; 40:36-46.
25. Huysamen C, Willment J A, Dennehy K M, Brown GD. CLEC9A is a novel activation C-type lectin-like receptor expressed on BDCA3+ dendritic cells and a subset of monocytes. J Biol. Chem. 2008; 283:16693-16701.
26. Caminschi I, Proietto A I, Ahmet F, et al. The dendritic cell subtype-restricted C-type lectin Clec9A is a target for vaccine enhancement. Blood. 2008; 112:3264-3273.
27. Sancho D, Mourao-Sa D, Joffre O P, et al. Tumor therapy in mice via antigen targeting to a novel, DC-restricted C-type lectin. J Clin Invest. 2008; 118:2098-2110.
28. Dudziak D, Kamphorst A O, Heidkamp G F, et al. Differential antigen processing by dendritic cell subsets in vivo. Science. 2007; 315:107-111.
29. Soares H, Waechter H, Glaichenhaus N, et al. A subset of dendritic cells induces CD4+ T cells to produce IFN-gamma by an IL-12-independent but CD70-dependent mechanism in vivo. J Exp Med. 2007; 204:1095-1106.
30. Tacken P J, de Vries I J, Gijzen K, et al. Effective induction of naive and recall T-cell responses by targeting antigen to human dendritic cells via a humanized anti-DC-SIGN antibody. Blood. 2005; 106:1278-1285.
31. Bozzacco L, Trumpfheller C, Siegal F P, et al. DEC-205 receptor on dendritic cells mediates presentation of HIV gag protein to CD8+ T cells in a spectrum of human MHC I haplotypes. Proc Natl Acad Sci USA. 2007; 104:1289-1294.
32. He L Z, Crocker A, Lee J, et al. Antigenic targeting of the human mannose receptor induces tumor immunity. J. Immunol. 2007; 178:6259-6267.
33. Bates E E, Fournier N, Garcia E, et al. APCs express DCIR, a novel C-type lectin surface receptor containing an immunoreceptor tyrosine-based inhibitory motif. J. Immunol. 1999; 163:1973-1983.
34. Meyer-Wentrup F, Benitez-Ribas D, Tacken P J, et al. Targeting DCIR on human plasmacytoid dendritic cells results in antigen presentation and inhibits IFN-alpha production. Blood. 2008; 111:4245-4253.
35. Lambert A A, Gilbert C, Richard M, Beaulieu A D, Tremblay M J. The C-type lectin surface receptor DCIR acts as a new attachment factor for HIV-1 in dendritic cells and contributes to trans- and cis-infection pathways. Blood. 2008; 112:1299-1307.
36. Kanazawa N, Okazaki T, Nishimura H, Tashiro K, Inaba K, Miyachi Y. DCIR acts as an inhibitory receptor depending on its immunoreceptor tyrosine-based inhibitory motif. J Invest Dermatol. 2002; 118:261-266.
37. Caux C, Vanbervliet B, Massacrier C, et al. CD34+ hematopoietic progenitors from human cord blood differentiate along two independent dendritic cell pathways in response to GM-CSF+TNF alpha. J Exp Med. 1996; 184:695-706.
38. Reddy M P, Kinney C A, Chaikin M A, et al. Elimination of Fc receptor-dependent effector functions of a modified IgG4 monoclonal antibody to human CD4. J. Immunol. 2000; 164:1925-1933.
39. Di Pucchio T, Chatterjee B, Smed-Sorensen A, et al. Direct proteasome-independent cross-presentation of viral antigen by plasmacytoid dendritic cells on major histocompatibility complex class I. Nat. Immunol. 2008; 9:551-557.
40. Meyer-Wentrup F, Cambi A, Joosten B, et al. DCIR is endocytosed into human dendritic cells and inhibits TLR8-mediated cytokine production. J Leukoc Biol. 2009; 85:518-525.
41. Fujikado N, Saijo S, Yonezawa T, et al. Dcir deficiency causes development of autoimmune diseases in mice due to excess expansion of dendritic cells. Nat. Med. 2008; 14:176-180.
42. Chen C H, Floyd H, Olson N E, et al. Dendritic-cell-associated C-type lectin 2 (DCAL-2) alters dendritic-cell maturation and cytokine production. Blood. 2006; 107:1459-1467.
43. Barrow A D, Trowsdale J. You say ITAM and I say ITIM, let's call the whole thing off: the ambiguity of immunoreceptor signalling. Eur J. Immunol. 2006; 36:1646-1653.
44. Wille-Reece U, Flynn B J, Lore K, et al. HIV Gag protein conjugated to a Toll-like receptor 7/8 agonist improves the magnitude and quality of Th1 and CD8+ T cell responses in nonhuman primates. Proc Natl Acad Sci USA. 2005; 102:15190-15194.
45. Wille-Reece U, Flynn B J, Lore K, et al. Toll-like receptor agonists influence the magnitude and quality of memory T cell responses after prime-boost immunization in nonhuman primates. J Exp Med. 2006; 203:1249-1258.
46. Caron G, Duluc D, Fremaux I, et al. Direct stimulation of human T cells via TLR5 and TLR7/8: flagellin and R-848 up-regulate proliferation and IFN-gamma production by memory CD4+ T cells. J. Immunol. 2005; 175:1551-1557.
47. Simone R, Floriani A, Saverino D. Stimulation of Human CD4 T Lymphocytes via TLR3, TLR5 and TLR7/8 Up-Regulates Expression of Costimulatory and Modulates Proliferation. Open Microbiol J. 2009; 3:1-8.
48. Adams S, O'Neill D W, Nonaka D, et al. Immunization of malignant melanoma patients with full-length NY-ESO-1 protein using TLR7 agonist imiquimod as vaccine adjuvant. J. Immunol. 2008; 181:776-784.
49. Delamarre L, Pack M, Chang H, Mellman I, Trombetta E S. Differential lysosomal proteolysis in antigen-presenting cells determines antigen fate. Science. 2005; 307:1630-1634.
50. Noy R, Eppel M, Haus-Cohen M, et al. T-cell receptor-like antibodies: novel reagents for clinical cancer immunology and immunotherapy. Expert Rev Anticancer Ther. 2005; 5:523-536.
51. Shulman M, Wilde C D, Kohler G. A better cell line for making hybridomas secreting specific antibodies. Nature. 1978; 276:269-270.
52. Trumpfheller C, Finke J S, Lopez C B, et al. Intensified and protective CD4+ T cell immunity in mice with anti-dendritic cell HIV gag fusion antibody vaccine. J Exp Med. 2006; 203:607-617.
53. Biddison W E, Turner R V, Gagnon S J, Lev A, Cohen C J, Reiter Y. Tax and M1 peptide/HLA-A2-specific Fabs and T cell receptors recognize nonidentical structural features on peptide/HLA-A2 complexes. J. Immunol. 2003; 171:3064-3074.
54. Regnault A, Lankar D, Lacabanne V, et al. Fcgamma receptor-mediated induction of dendritic cell maturation and major histocompatibility complex class I-restricted antigen presentation after immune complex internalization. J Exp Med. 1999; 189:371-380.

What is claimed is:

1. An immunostimulatory composition for generating an immune response, for a prophylaxis, a therapy or any combination thereof in a human or animal subject comprising:
one or more anti-dendritic cell (DC)-specific antibodies or fragments thereof loaded or chemically coupled with one or more antigenic peptides, wherein the antigenic peptides are representative of one or more epitopes of the one or more antigens implicated or involved in a disease or a condition against which the immune response, the prophylaxis, the therapy, or any combination thereof is desired;
at least one Toll-Like Receptor (TLR) agonist which is selected from the group consisting of TLR1, TLR2, TLR3, TLR4, TLR5, TLR6, TLR7, and TLR8 agonists; and
a pharmaceutically acceptable carrier, wherein the conjugate and agonist are each comprised in an amount such that, in combination with the other, are effective to produce the immune response, for prophylaxis, for therapy or any combination thereof in the human or animal subject in need of immunostimulation.

2. The composition of claim 1, wherein the composition comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines, or combinations and modifications thereof.

3. The composition of claim 1, wherein the anti-DC-specific antibody or fragment is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

4. The composition of claim 1, wherein the anti-DC-specific antibody is an anti-DCIR antibody selected from ATCC Accession No. PTA 10246 or PTA 10247.

5. The composition of claim 1, wherein the antigenic peptides comprise human immunodeficiency virus (HIV) antigens and gene products selected from the group consisting of gag, pol, and env genes, the Nef protein, reverse transcriptase, string of HIV peptides (Hipo5), PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, or combinations and modifications thereof.

6. The composition of claim 1, wherein the antigenic peptides are cancer peptides are selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma, and leukemia.

7. The composition of claim 6, wherein the tumor associated antigens are selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67.

8. The composition of claim 1, wherein the anti-DC-specific antibody is humanized.

9. The composition of claim 1, wherein the composition is administered to the human or animal subject by an oral route, a nasal route, topically, or as an injection, wherein the injection is selected from the group consisting of subcutaneous, intravenous, intraperitoneal, intramuscular, and intravenous.

10. A vaccine comprising
one or more anti-dendritic cell (DC)-specific antibodies or fragments thereof loaded or chemically coupled with one or more antigenic peptides;
at least one Toll-Like Receptor (TLR) agonist, wherein the TLR agonist is selected from the group consisting of TLR1, TLR2, TLR3, TLR4, TLR5, TLR6, TLR7, and TLR8 agonists; and
one or more optional pharmaceutically acceptable carriers and adjuvants, wherein the antibody and the agonist are each comprised in an amount such that, in combination with the other, are effective to produce an immune response, for a prophylaxis, a therapy, or any combination thereof in a human or an animal subject.

11. The vaccine of claim 10, wherein the vaccine comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines, or combinations and modifications thereof.

12. The vaccine of claim 10, wherein the anti-DC-specific antibody or fragment is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

13. The vaccine of claim 10, wherein the anti-DC-specific antibody is an anti-DCIR antibody selected from ATCC Accession No. PTA 10246 or PTA 10247.

14. The vaccine of claim 10, wherein the antigenic peptides comprise human immunodeficiency virus (HIV) antigens and gene products selected from the group consisting of gag, pol, and env genes, the Nef protein, reverse transcriptase, string of HIV peptides (Hipo5), PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, or combinations and modifications thereof.

15. The vaccine of claim 10, wherein the antigenic peptide is a cancer peptide comprising tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67.

16. The vaccine of claim 10, wherein the anti-DC-specific antibody is humanized.

17. The vaccine of claim 10, wherein the composition is administered to the human or animal subject by an oral route, a nasal route, topically, or as an injection.

18. A method for increasing effectiveness of antigen presentation by an antigen presenting cell (APC) comprising:
isolating and purifying one or more anti-dendritic cell (DC)-specific antibodies or fragments thereof;
loading or chemically coupling one or more native or engineered antigenic peptides to the DC-specific antibody to form an antibody-antigen conjugate;
adding at least one Toll-Like Receptor (TLR) agonist selected from the group consisting of TLR1, TLR2, TLR3, TLR4, TLR5, TLR6, TLR7, and TLR8 agonists to the conjugate; and
contacting the APC with the conjugate and the TLR agonist, wherein the antibody-antigen complex is processed and presented for T cell recognition.

19. The method of claim 18, further comprising the optional steps of:
adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the antibody-antigen conjugate and the TLR agonist prior to contacting the antigen presenting cells; and
measuring a level of one or more agents selected from the group consisting of IFN-γ, TNF-α, IL-12p40, IL-4, IL-5, and IL-13, wherein a change in the level of the one or more agents is indicative of the increase in the effectiveness antigen presentation by the antigen presenting cell.

20. The method of claim 18, wherein the APC comprises a dendritic cell (DC).

21. The method of claim 18, wherein the anti-DC-specific antibody or fragment is selected from an antibody that specifically binds to dendritic cell immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

22. The method of claim 18, wherein the anti-DC-specific antibody is an anti-DCIR antibody selected from ATCC Accession No. PTA 10246 or PTA 10247.

23. The method of claim 18, wherein the antigenic peptides comprise human immunodeficiency virus (HIV) antigens and gene products selected from the group consisting of gag, pol, and env genes, the Nef protein, reverse transcriptase, string of HIV peptides (Hipo5), PSA-tetramer, a HIVgag-derived p24-PLA HIV gag p24 (gag), and other HIV components, hepatitis viral antigens, influenza viral antigens and peptides selected from the group consisting of hemagglutinin, neuraminidase, Influenza A Hemagglutinin HA-1 from a H1N1 Flu strain, HLA-A201-FluMP (58-66) peptide tetramer, and Avian Flu (HA5-1), dockerin domain from C. thermocellum, measles viral antigens, rubella viral antigens, rotaviral antigens, cytomegaloviral antigens, respiratory syncytial viral antigens, herpes simplex viral antigens, varicella zoster viral antigens, Japanese encephalitis viral antigens, rabies viral antigens, or combinations and modifications thereof.

24. The method of claim 18, wherein the antigenic peptide is a cancer peptide comprising tumor associated antigens selected from CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67.

25. The method of claim 18, wherein the anti-DC-specific antibody is humanized.

26. A vaccine comprising:
an anti-dendritic cell immunoreceptor (DCIR) monoclonal antibody conjugate, wherein the conjugate comprises the DCIR monoclonal antibody or a fragment thereof loaded or chemically coupled with one or more antigenic peptides;
at least one Toll-Like Receptor (TLR) agonist selected from the group consisting of TLR1, TLR2, TLR3, TLR4, TLR5, TLR6, TLR7, and TLR8 agonists; and
one or more optional pharmaceutically acceptable carriers and adjuvants, wherein the conjugate and agonist are each comprised in an amount such that, in combination with the other, are effective to produce an immune response, for a prophylaxis, a therapy or any combination thereof against one or more diseases or conditions in a human or an animal subject in need thereof.

27. The vaccine of claim 26, wherein the vaccine is adapted for use in a treatment, a prophylaxis, or a combination thereof against one or more diseases or conditions selected from influenza, HIV, cancer, and any combinations thereof in a human subject.

28. The vaccine of claim 26, wherein the one or more antigenic peptides is a FluMP peptide comprising SEQ ID NO: 1.

29. The vaccine of claim 26, wherein the one or more antigenic peptides is a MART-1 peptide comprising SEQ ID NO: 2.

30. The vaccine of claim 26, wherein the one or more antigenic peptides is a HIV gagp24 peptide comprising SEQ ID NO: 3.

31. The vaccine of claim 26, wherein the vaccine comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines, or combinations and modifications thereof.

32. The vaccine of claim 26, wherein vaccine further comprises an optional anti-DC-specific antibody or a fragment thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

33. A method for a treatment, a prophylaxis, or a combination thereof against one or more diseases or conditions in a human subject comprising the steps of:
identifying the human subject in need of the treatment, the prophylaxis or a combination thereof against the one or more diseases or conditions; and
administering a vaccine composition comprising:
an anti-dendritic cell immunoreceptor (DCIR) monoclonal antibody conjugate, wherein the conjugate comprises the DCIR monoclonal antibody or a fragment thereof loaded or chemically coupled with one or more antigenic peptides, wherein the antigenic peptides are representative of one or more epitopes of the one or more antigens implicated or involved in the one or more diseases or conditions against which the prophylaxis, the therapy, or both is desired;
at least one Toll-Like Receptor (TLR) agonist selected from the group consisting of TLR1, TLR2, TLR3, TLR4, TLR5, TLR6, TLR7, and TLR8 agonists; and
one or more optional pharmaceutically acceptable carriers and adjuvants, wherein the conjugate and agonist are each comprised in an amount such that, in combination with the other, are effective to produce an immune response, for the prophylaxis, the therapy or any combination thereof against the one or more diseases or conditions in the human subject.

34. The method of claim 33, wherein the one or more diseases or conditions are selected from the group consisting of influenza, cancer, HIV, or any combinations thereof.

35. The method of claim 34, wherein the cancers is selected from the group consisting of leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

36. The method of claim 33, wherein the one or more antigenic peptides is a FluMP peptide comprising SEQ ID NO: 1.

37. The method of claim 33, wherein the one or more antigenic peptides is a MART-1 peptide comprising SEQ ID NO: 2.

38. The method of claim 33, wherein the one or more antigenic peptides is a HIV gagp24 peptide comprising SEQ ID NO: 3.

39. The method of claim 33, wherein the vaccine comprises one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines, or combinations and modifications thereof.

40. The method of claim 33, wherein vaccine further comprises an optional anti-DC-specific antibody or a fragment thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

41. The method of claim 33, wherein the vaccine is administered to the human subject by an oral route, a nasal route, topically, or as an injection.

42. A method for increasing effectiveness of antigen presentation by one or more dendritic cells (DCs) in a human subject comprising the steps of:
isolating one or more DCs from the human;
exposing the isolated DCs to activating amounts of a composition or a vaccine comprising:
an anti-dendritic cell immunoreceptor (DCIR) monoclonal antibody conjugate, wherein the conjugate comprises the DCIR monoclonal antibody or a fragment thereof loaded or chemically coupled with one or more antigenic peptides;
at least one Toll-Like Receptor (TLR) agonist which is selected from the group consisting of TLR1, TLR2, TLR3, TLR4, TLR5, TLR6, TLR7, and TLR8 agonists;
and a pharmaceutically acceptable carrier to form an activated DC complex; and
reintroducing the activated DC complex into the human subject.

43. The method of claim 42, further comprising the optional step of measuring a level of one or more agents selected from the group consisting of IFN-γ, TNF-α, IL-12p40, IL-4, IL-5, and IL-13, wherein a change in the level of the one or more agents is indicative of the increase in the effectiveness of the one or more DCs.

44. The method of claim 42, further comprising the step of adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the conjugate and the TLR agonist prior to exposing the DCs.

45. The method of claim 42, further comprising the step of adding one or more optional anti-DC-specific antibodies or fragments thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

46. The method of claim 42, wherein the antigenic peptides comprise one or more human immunodeficiency virus (HIV) antigens and gene products, one or more cancer peptide and tumor associated antigens, or both.

47. A method of providing immunostimulation by activation of one or more dendritic cells (DCs) to a human subject for a prophylaxis, a therapy, or a combination thereof against one or more viral, bacterial, fungal, parasitic, protozoal, parasitic diseases and allergic disorders comprising the steps of:
identifying the human subject in need of immunostimulation for the prophylaxis, the therapy, or a combination thereof against the one or more viral, bacterial, fungal, parasitic, protozoal, parasitic diseases and allergic disorders;
isolating one or more DCs from the human subject;
exposing the isolated DCs to activating amounts of a composition or a vaccine comprising an anti-dendritic cell immunoreceptor (DCIR) monoclonal antibody conjugate, wherein the conjugate comprises the DCIR monoclonal antibody or a fragment thereof loaded or chemically coupled with one or more antigenic peptides, at least one Toll-Like Receptor (TLR) agonist selected from the group consisting of TLR1, TLR2, TLR3, TLR4, TLR5, TLR6, TLR7, and TLR8 agonists and a pharmaceutically acceptable carrier to form an activated DC complex; and
reintroducing the activated DC complex into the human subject.

48. The method of claim 47, further comprising the optional step of measuring a level of one or more agents selected from the group consisting of IFN-γ, TNF-α, IL-12p40, IL-4, IL-5, and IL-13, wherein a change in the level of the one or more agents is indicative of the immunostimulation.

49. The method of claim 47, further comprising the step of adding one or more optional agents selected from the group consisting of an agonistic anti-CD40 antibody, an agonistic anti-CD40 antibody fragment, a CD40 ligand (CD40L) polypeptide, a CD40L polypeptide fragment, anti-4-1BB antibody, an anti-4-1BB antibody fragment, 4-1BB ligand polypeptide, a 4-1BB ligand polypeptide fragment, IFN-γ, TNF-α, type 1 cytokines, type 2 cytokines or combinations and modifications thereof to the conjugate and the TLR agonist prior to exposing the DCs.

50. The method of claim 47, further comprising the step of adding one or more optional anti-DC-specific antibodies or fragments thereof selected from antibodies specifically binding to MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor, LOX-1, and ASPGR.

51. The method of claim 47, wherein the antigenic peptide comprises bacterial antigens selected from pertussis toxin, filamentous hemagglutinin, pertactin, FIM2, FIM3, adenylate cyclase and other pertussis bacterial antigen components, diptheria bacterial antigens, diptheria toxin or toxoid, other diptheria bacterial antigen components, tetanus bacterial antigens, tetanus toxin or toxoid, other tetanus bacterial antigen components, streptococcal bacterial antigens, gram-negative bacilli bacterial antigens, Mycobacterium tuberculosis bacterial antigens, mycolic acid, heat shock protein 65 (HSP65), Helicobacter pylori bacterial antigen components; pneumococcal bacterial antigens, haemophilus influenza bacterial antigens, anthrax bacterial antigens, and rickettsiae bacterial antigens.

52. The method of claim 47, wherein the antigenic peptide comprises fungal antigens selected from candida fungal antigen components, histoplasma fungal antigens, cryptococcal fungal antigens, coccidiodes fungal antigens and tinea fungal antigens.

53. The method of claim 47, wherein the antigenic peptide comprises protozoal and parasitic antigens antigens selected from plasmodium falciparum antigens, sporozoite surface antigens, circumsporozoite antigens, gametocyte/gamete surface antigens, blood-stage antigen pf 155/RESA, toxoplasma, schistosomae antigens, leishmania major and other leishmaniae antigens and trypanosoma cruzi antigens.

54. The method of claim 47, wherein the antigenic peptide comprises antigens involved in autoimmune diseases, allergy, and graft rejection selected from diabetes, diabetes mellitus, arthritis, multiple sclerosis, myasthenia gravis, systemic lupus erythematosis, autoimmune thyroiditis, dermatitis, psoriasis, Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

55. The method of claim 47, wherein the antigenic peptide comprises antigens involved in allergic disorders selected from Japanese cedar pollen antigens, ragweed pollen antigens, rye grass pollen antigens, animal derived antigens, dust mite antigens, feline antigens, histocompatiblity antigens, and penicillin and other therapeutic drugs.

56. The method of claim 47, wherein the DC-specific antibody is humanized.
